(19) United States
(12) Patent Application Publication
(10) Pub. No.: US 2025/0376647 A1
(43) Pub. Date: Dec. 11, 2025

(54) PROCESS FOR PREPARING DISSOLVABLE UNIT DOSE SHEET ARTICLES

(71) Applicant: The Procter & Gamble Company, Cincinnati, OH (US)

(72) Inventors: Carl David MAC NAMARA, Beijing (CN); Hong Sing TAN, Beijing (CN)

(21) Appl. No.: 19/229,383

(22) Filed: Jun. 5, 2025

(30) Foreign Application Priority Data
Jun. 6, 2024 (WO) PCT/CN2024/097822

(51) Int. Cl. C11D 17/04; A45D 37/00; A61K 8/02; A61K 9/70; B65B 9/02; B65B 51/10; C11D 1/12; C11D 1/14; C11D 1/22; C11D 1/72; C11D 1/83; C11D 3/32; C11D 3/37; C11D 3/39; C11D 3/43

(52) U.S. Cl. CPC C11D 17/044; A45D 37/00; A61K 8/0208; A61K 9/7007; B65B 9/023; B65B 51/10; C11D 1/83; C11D 3/32; C11D 3/3788; C11D 3/3942; C11D 3/43; C11D 17/043; C11D 1/123; C11D 1/146; C11D 1/22; C11D 1/72

(57) ABSTRACT

The present invention relates to a process for preparing a dissolvable unit dose sheet article.

### FIELD OF THE INVENTION

The present invention relates to a process for preparing a dissolvable unit dose sheet article.

### BACKGROUND OF THE INVENTION

In the area of detergent products, water-soluble unit dose articles are liked by consumers due to their convenience and case of use. Consumers also like the fact that they do not need to measure a detergent dose and so this eliminates accidental spillage during the dosing operation. Accidental dosage can be messy and inconvenient. Various water-soluble unit dose articles have been developed, including dissolvable porous solid sheet articles comprising a water-soluble polymeric structurant and a surfactant or other ingredient. In a typical process for preparing a dissolvable porous solid sheet, a pre-mixture of raw materials is first formed, which is vigorously aerated and then heat-dried in a batch process or a continuous process to form the porous sheets.

However, some active ingredients that are not suitable for processing into the sheets due to thermal stability or deactivation upon contact with water may be applied as a loading composition between layers of the flexible dissolvable sheet article. Such loading composition can be in a form of a paste or solid particles. The introduction of the loading composition may lead to some defects including accidental leakage during the storage and/or shipment and difficulties for edge-scaling.

Heat-compressing processes, for example edge-sealing, have been used in sealing of plastic packaging or some other products. For example, heating and/or pressure can be applied onto the edge of two layers of plastic film so as to at least partially melt the film, and then, the edge is sealed after the cooling of the film. Some previous studies have tried such process in sealing of dissolvable unit dose sheet articles. However, the previous studies found that, unlike the plastic packaging, such heat-compressing process cannot provide a desirable sealing for dissolvable unit dose sheet articles containing a loading composition probably due to inherent properties of dissolvable porous solid sheet, e.g., intolerance of high temperature and high pressure, the porous structure and the like.

Thus, a need still exists for a process that results in a desired sealing and/or improved leakage performance.

### SUMMARY OF THE INVENTION

The inventors of the present invention surprisingly found that the heat-compressing process can work well when the dissolvable porous solid sheet has a relatively high compressibility.

In one aspect, the present invention relates to a process for preparing a dissolvable unit dose sheet article, comprising the steps of: a) providing a first flexible, dissolvable, porous sheet, a second flexible, dissolvable, porous sheet and a loading composition in a form of paste or powders in which each of the first and second sheets comprises a water-soluble polymer and a surfactant; wherein each of the first and the second sheets is characterized by: (1) a Percent Open Cell Content of from 80% to 100%, (2) an Overall Average Pore Size of from 100 μm to 2000 μm, and (3) a Compressibility of less than 90,000N/m²; b) applying the loading composition on a surface of the first sheet; c) arranging the first and second sheets into a stack so that the loading composition is contained between the first and second sheets; and d) heat-compressing said stack of sheets to form the dissolvable unit dose article.

In some embodiments, each of the first and the second sheets is characterized by a Compressibility of from 1,000 N/m² to 90,000 N/m², preferably from 2,000 N/m² to 80,000 N/m², more preferably from 3,000 N/m² to 70,000 N/m², most preferably from 4,000 N/m² to 60,000 N/m², e.g. 4,000 N/m², 5,000 N/m², 10,000 N/m², 20,000 N/m², 30,000 N/m², 40,000 N/m², 50,000 N/m², or any ranges therebetween.

In some embodiments, the heat-compressing is performed under a temperature of from 50° C. to 200° C., preferably from 70° C. to 180° C., more preferably from 90° C. to 170° C., a pressure of from 100 psi to 20000 psi, preferably from 1000 psi to 10000 psi, more preferably from 1500 psi to 5000 psi, e.g. 1000 psi, 1500 psi, 2000 psi, 2500 psi, 3000 psi, 3500 psi, 4000 psi, 5000 psi, 8000 psi or any ranges therebetween, and a contacting time of from 0.02 s to 10 s, preferably from 0.05 s to 5s, more preferably from 0.05 s to 1 s, e.g. 0.05 s, 0.1 s, 0.15 s, 0.2 s, 0.3 s, 0.4 s, 0.5 s, 0.8 s, 1 s or any ranges therebetween.

In some embodiments, the heat-compressing is performed on at least 5%, preferably from 5% to 100%, more preferably from 20% to 100%, e.g. 20%, 30%, 40%, 50%, 60%, 70%, 80%, 90%, or any ranges therebetween, of the surface area of the sheet.

In some embodiments, the heat-compressing is selected from the group consisting of edge-sealing, embossing and any combinations thereof.

In some embodiments, the loading composition is in a form of non-aqueous paste and comprises a non-aqueous liquid carrier, solid particles and a polyalkylene polymer. Preferably, wherein said non-aqueous paste comprises: 1) from 1% to 99%, preferably from 5% to 70%, more preferably from 10% to 60%, of a non-aqueous liquid carrier by total weight of said non-aqueous paste; and/or 2) from 1% to 99%, preferably from 10% to 80%, more preferably from 30% to 75%, of solid particles by total weight of said non-aqueous paste; and/or 3) from 0.5% to 50%, preferably from 0.8% to 30%, more preferably from 1% to 20%, of a polyalkylene polymer by total weight of said non-aqueous paste.

In some embodiments, said non-aqueous liquid carrier is selected from the group consisting of polyethylene glycol, polypropylene glycol, silicone, fatty acid, perfume oil, a non-ionic surfactant, an organic solvent and any combinations thereof, preferably wherein said non-aqueous liquid carrier comprises a non-ionic surfactant that is preferably selected from the group consisting of C₆-C₂₀ linear or branched alkylalkoxylated alcohols (AA) having a weight average degree of alkoxylation ranging from 5 to 15.

In some embodiments, said solid particles comprise an oxidative dye compound, a pH modifier and/or a buffering agent, a radical scavenger, a chelant, a warming active, a color indicator, an anionic surfactant, an enzyme, a bleaching agent, an effervescent system and any combinations thereof, preferably, wherein said solid particles comprises C₆-C₂₀ linear alkylbenzene sulphonate (LAS) surfactant, percarbonate salts, perborate salts, persulfate salts, tetraacetylethylenediamine (TAED), oxybenzene sulphonates, caprolactams, or any combinations thereof.

In some embodiments, said polyalkylene polymer is selected from a group consisting of polyalkylene imine polymer, polyalkylene oxide polymer and any combinations thereof, preferably wherein said polyalkylene polymer is a polyalkylene graft copolymer comprising a) polyalkylene oxide component as a graft base, and b) polyvinyl ester component as side chains, and/or c) polyvinylpyrrolidone as side chains. In some embodiments, the loading composition is in a form of powders which are characterized by a bulk density of from 250 g/l to 500 g/l, e.g., from 300 g/l to 500 g/l, from 350 g/l to 500 g/l. Preferably, the powders are characterized by a mean particle size of from about 200 to about 600 microns, preferably from about 300 to about 500 microns.

In some embodiments, said powders comprises an anionic surfactant which is preferably selected from the group consisting of C₆-C₂₀ linear alkylbenzene sulfonate (LAS), a C₆-C₂₀ linear or branched alkylalkoxy sulfates (AAS) having a weight average degree of alkoxylation ranging from 0.5 to 10, a C₆-C₂₀ linear or branched alkyl sulfates (AS) and any combinations thereof.

In some embodiments, an aqueous liquid (e.g. water) is sprayed on a surface of the second sheet before the Step c) in which the surface of the second sheet is located to be adjacent to the first sheet.

In some embodiments, the process is a continuous process which is performed on a conveying belt.

In some embodiments, the process further comprises: providing one or more additional flexible, dissolvable, porous sheets onto the stack of the first and the second sheets before heat-compressing, wherein each of the additional sheets is characterized by: (1) a Percent Open Cell Content of from 80% to 100%, (2) an Overall Average Pore Size of from 100 μm to 2000 μm, and (3) a Compressibility of less than 90,000N/m².

In some embodiments, Step b) is conducted by a loading unit comprising a nozzle and a flattening mechanism or a dispenser and a spreading roller.

In some embodiments, Step c) is conducted by one or more rollers.

In some embodiments, Step d) is conducted by an edge-sealing roller and optionally a transferring roller.

In some embodiments, Step d) is conducted by an embossing roller and a cutting roller as well as optionally a transferring roller.

In some embodiments, the weight ratio of the water-soluble sheets and the loading composition in the dissolvable unit dose article is between 1000 and 0.1, preferably between 100 and 0.15, more preferably between 20 and 0.2, e.g. 20, 15, 10, 5, 3, 2, 1, 0.8, 0.7, 0.6, 0.5, 0.4, 0.3 or any ranges therebetween.

In some embodiments, each of the sheets is characterized by:
a Percent Open Cell Content of from 85% to 100%, preferably from 90% to 100%; and/or
an Overall Average Pore Size of from 150 μm to 1000 μm, preferably from 200 μm to 600 μm; and/or
an Average Cell Wall Thickness of from 5 μm to 200 μm, preferably from 10 μm to 100 μm, more preferably from 10 μm to 80 μm; and/or
a final moisture content of from 0.5% to 25%, preferably from 1% to 20%, more preferably from 3% to 10%, by weight of said solid sheet article; and/or
a thickness of from 0.6 mm to 3.5 mm, preferably from 0.7 mm to 3 mm, more preferably from 0.8 mm to 2 mm, most preferably from 1 mm to 2 mm; and/or
a basis weight of from 50 grams/m² to 500 grams/m², preferably from 150 grams/m² to 450 grams/m², more preferably from 250 grams/m² to 400 grams/m²; and/or
a density of from 0.05 grams/cm³ to 0.5 grams/cm³, preferably from 0.06 grams/cm³ to 0.4 grams/cm³, more preferably from 0.07 grams/cm³ to 0.2 grams/cm³, most preferably from 0.08 grams/cm³ to 0.15 grams/cm³; and/or
a Specific Surface Area of from 0.03 m²/g to 0.25 m²/g, preferably from 0.04 m²/g to 0.22 m²/g, more preferably from 0.05 m²/g to 0.2 m²/g, most preferably from 0.1 m²/g to 0.18 m²/g.

In another aspect, the present invention relates to a product in the form of a dissolvable unit dose sheet article, wherein the product is selected from the group consisting of laundry detergent products, fabric softening products, hand cleansing products, hair shampoo or other hair treatment products, body cleansing products, shaving preparation products, dish cleaning products, personal care substrates containing pharmaceutical or other skin care actives, moisturizing products, sunscreen products, beauty or skin care products, deodorizing products, oral care products, feminine cleansing products, baby care products, fragrance-containing products and any combinations thereof, wherein the product comprises a plurality of water-soluble sheets arranged in a stack, wherein each of the water-soluble sheets comprises a water-soluble polymer and a surfactant, wherein each of the water-soluble sheets is characterized by: (i) a Percent Open Cell Content of from 80% to 100%; (ii) an Overall Average Pore Size of from 100 μm to 2000 μm; and (3) a Compressibility of less than 90,000N/m²; wherein the product has a sealed edge and/or an embossing. Particularly, said article is prepared by the process according to the present disclosure.

In another aspect, the present invention relates to a dissolvable unit dose sheet article comprising: two or more flexible, porous, dissolvable solid sheets; a loading composition in a form of paste or powders contained within said two or more sheets; and an edge seal being generally positioned along at least a portion of the perimeter of the article; wherein each of the sheets comprises a water-soluble polymer and a surfactant; wherein each of the sheets is characterized by: (1) a Percent Open Cell Content of from 80% to 100%, (2) an Overall Average Pore Size of from 100 μm to 2000 μm, and (3) a Compressibility of less than 90,000N/m².

In another aspect, the present invention relates to a system for preparing dissolvable unit dose sheet articles according to the present disclosure, wherein the system comprises: a belt conveyor on which two or more flexible, porous, dissolvable solid sheets are sequentially fed; a loading unit which is configured to load a loading composition within said two or more sheets, comprising a nozzle and a flattening mechanism or a dispenser and a spreading roller; a heat-compressing unit which is configured to seal edges of the unit dose articles which may be preferably an edge-scaling unit or an embossing unit.

It is advantageous that the process according to the present disclosure can provide a desirable scaling for dissolvable unit dose sheet articles containing a loading composition. Surprisingly, the dissolvable porous solid sheets can be bonded together even when the loading composition is present in the bonding location.

It is further advantageous that the process according to the present disclosure can provide an improved leakage performance.

### BRIEF DESCRIPTION OF THE DRAWINGS

FIG. 1 shows an exemplary dissolvable unit dose article containing a loading composition in a form of paste.

FIG. 2 shows another exemplary dissolvable unit dose article having an edge seal and an embossed pattern.

FIG. 3 shows a photo of edge-sealed dissolvable unit dose article containing a loading composition in a form of paste and another loading composition in a form of powders which achieves an excellent leakage performance.

FIG. 4 shows an exemplary system for making a dissolvable unit dose article containing a loading composition in a form of paste.

FIG. 5 shows an exemplary tooling used to form the edge seal of the article.

FIG. 6 shows another exemplary system for making a dissolvable unit dose article containing a loading composition in a form of paste and another loading composition in a form of powders.

FIG. 7 shows another exemplary system for making a dissolvable unit dose article.

### DETAILED DESCRIPTION OF THE INVENTION

In all embodiments of the present invention, all percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

### I. DEFINITIONS

The term “flexible” as used herein refers to the ability of an article to withstand stress without breakage or significant fracture when it is bent at 90° along a center line perpendicular to its longitudinal direction. Preferably, such article can undergo significant elastic deformation and is characterized by a Young's Modulus of no more than 5 GPa, preferably no more than 1 GPa, more preferably no more than 0.5 GPa, most preferably no more than 0.2 GPa.

The term “dissolvable” as used herein refers to the ability of an article to completely or substantially dissolve in a sufficient amount of deionized water at 20° C. and under the atmospheric pressure within eight (8) hours without any stirring, leaving less than 5 wt % undissolved residues.

The term “solid” as used herein refers to the ability of an article to substantially retain its shape (i.e., without any visible change in its shape) at 20° C. and under the atmospheric pressure, when it is not confined and when no external force is applied thereto.

The term “sheet” as used herein refers to a non-fibrous structure having a three-dimensional shape, i.e., with a thickness, a length, and a width, while the length-to-thickness aspect ratio and the width-to-thickness aspect ratio are both at least about 5:1, and the length-to-width ratio is at least about 1:1. Preferably, the length-to-thickness aspect ratio and the width-to-thickness aspect ratio are both at least about 10:1, more preferably at least about 15:1, most preferably at least about 20:1; and the length-to-width aspect ratio is preferably at least about 1.2:1, more preferably at least about 1.5:1, most preferably at least about 1.618:1.

As used herein, the term “continuous” process refers to a manufacturing method where the production of a product is ongoing without a defined start or endpoint. The term “batch” process refers to a manufacturing method where a specific quantity of goods are made in a single production run. It has a defined start and endpoint, meaning the process is completed once the batch has been produced.

As used herein, the term “bottom surface” refers to a surface of the flexible, porous, dissolvable solid sheet article of the present invention that is immediately contacting a supporting surface upon which the sheet of aerated wet pre-mixture is placed during the drying step, while the term “top surface” refers to a surface of the sheet article that is opposite to the bottom surface. Further, such solid sheet article can be divided into three (3) regions along its thickness, including a top region that is adjacent to its top surface, a bottom region that is adjacent to its bottom surface, and a middle region that is located between the top and bottom regions. The top, middle, and bottom regions are of equal thickness, i.e., each having a thickness that is about ⅓ of the total thickness of the sheet article.

The term “open celled foam” or “open cell pore structure” as used herein refers to a solid, interconnected, polymer-containing matrix that defines a network of spaces or cells that contain a gas, typically a gas (such as air), without collapse of the foam structure during the drying process, thereby maintaining the physical strength and cohesiveness of the solid. The interconnectivity of the structure may be described by a Percent Open Cell Content, which is measured by Test 3 disclosed hereinafter.

The term “water-soluble” as used herein refers to the ability of a sample material to completely dissolve in or disperse into water leaving no visible solids or forming no visibly separate phase, when at least about 25 grams, preferably at least about 50 grams, more preferably at least about 100 grams, most preferably at least about 200 grams, of such material is placed in one liter (1 L) of deionized water at 20° C. and under the atmospheric pressure with sufficient stirring.

The term “aerate”, “aerating” or “aeration” as used herein refers to a process of introducing a gas into a liquid or pasty composition by mechanical and/or chemical means.

The term “heating direction” as used herein refers to the direction along which a heat source applies thermal energy to an article, which results in a temperature gradient in such article that decreases from one side of such article to the other side. For example, if a heat source located at one side of the article applies thermal energy to the article to generate a temperature gradient that decreases from the one side to an opposing side, the heating direction is then deemed as extending from the one side to the opposing side. If both sides of such article, or different sections of such article, are heated simultaneously with no observable temperature gradient across such article, then the heating is carried out in a non-directional manner, and there is no heating direction.

The term “substantially opposite to” or “substantially offset from” as used herein refers to two directions or two lines having an offset angle of 90° or more therebetween.

The term “substantially aligned” or “substantial alignment” as used herein refers to two directions or two lines having an offset angle of less than 90° therebetween.

The term “age” or “aging” as used herein refers to a process of maintaining an aerated wet mixture or pre-mixture for a while without further introducing a significant amount of gas. Preferably, the aging may be conducted under the conditions of being essentially free of mechanical energy input and/or being essentially free of heat input. More preferably, the aging may be conducted under the ambient temperature without any stirring.

The term “heat-compressing” as used herein refers to a process of applying both heat and pressure on materials (e.g. flexible, porous, dissolvable solid sheets according to the present disclosure) which may cause a partial melting of the materials and then re-solidification. The term “edge-sealing” as used herein refers to a particular heat-compressing in which the heat and pressure are applied only on the area which is close to the edge of materials so that adjacent layers may bond together. The term “embossing” as used herein refers to a particular heat-compressing in which the heat and pressure are applied on some specific area of materials so as to form a three-dimension pattern. Particularly, embossing may be applied at discrete points across the material, e.g. the surface of dissolvable unit dose sheet articles. In the context of the present disclosure, the inventors of the present invention surprisingly found that heat-compressing including edge-scaling and embossing can provide a good sealing for dissolvable unit dose sheet articles according to the present disclosure. In some embodiments, either edge-scaling or embossing is included in the process of preparing a dissolvable unit dose sheet article. In some other embodiments, both edge-sealing and embossing are included in the process of preparing a dissolvable unit dose sheet article.

The term “essentially free of” or “essentially free from” means that the indicated material is at the very minimal not deliberately added to the composition or product, or preferably not present at an analytically detectible level in such composition or product. It may include compositions or products in which the indicated material is present only as an impurity of one or more of the materials deliberately added to such compositions or products.

The test methods disclosed in the Test Methods Section of the present application should be used to determine the respective values of the parameters of Applicants' inventions.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### II. OVERVIEW OF PROCESSES FOR MAKING UNIT DOSE ARTICLES

The dissolvable porous solid sheet according to the present disclosure can be made by using known methods. For example, WO2010077627 discloses a batch process for forming porous sheets with open-celled foam (OCF) structures. WO2012138820 discloses a similar process as that of WO2010077627, except that continuous drying of the aerated wet pre-mixture is achieved by using, e.g., an impingement oven (instead of a convection oven or a microwave oven). Furthermore, WO2021/102935 discloses another drying process for making the porous sheets. A typical method for making flexible, porous, dissolvable solid sheets may comprise the steps of: (a) forming a pre-mixture containing raw materials (e.g., the water-soluble polymer, active ingredients such as surfactants, and optionally a plasticizer) dissolved or dispersed in water or a suitable solvent, which is characterized by a viscosity of from about 1,000 cps to about 25,000 cps measured at about 40° C. and 1 s⁻¹; (b) aerating the pre-mixture (e.g., by introducing a gas into the wet slurry) to form an aerated wet pre-mixture; (c) forming the aerated wet pre-mixture into a sheet having opposing first and second sides; and (d) drying the formed sheet for a drying time of from 1 minute to 60 minutes at a temperature from 70° C. to 200° C. along a heating direction that forms a temperature gradient decreasing from the first side to the second side of the formed sheet, wherein the heating direction is substantially offset from the gravitational direction for more than half of the drying time, i.e., the drying step is conducted under heating along a mostly “anti-gravity” heating direction. Such a mostly “anti-gravity” heating direction can be achieved by various means, which include but are not limited to the bottom conduction-based heating/drying arrangement and the rotary drum-based heating/drying arrangement.

The dissolvable porous solid sheet can be prepared by the continuous process comprising: a) preparing a wet pre-mixture comprising a water-soluble polymer and a surfactant and having a viscosity of from 1,000 cps to 25,000 cps measured at 40° C. and 1 s⁻¹; b) aerating the wet pre-mixture to form an aerated wet pre-mixture having a density of from 0.05 to 0.5 g/ml; c) forming the aerated wet pre-mixture into a sheet having a top side and a bottom side, for example by extruding the aerated wet pre-mixture; and d) drying the formed sheet of aerated wet pre-mixture on a conveying belt with the bottom side of the formed sheet contacting the conveying belt.

The drying of the formed aerated wet pre-mixture according to the present application is a step-wise process. Particularly, the conveying belt is configured to sequentially pass through multiple heating zones with heating temperatures ranging from 70° C. to 200° C.; wherein said multiple heating zones comprises a first heating zone and a second heating zone which is located downstream of said first heating zone. More particularly, said first heating zone is configured to simultaneously heat the top and bottom sides of said formed sheet at a first top heating temperature (Tₜ₁) and a first bottom heating temperature (T_{b1}) for a first heating duration of from 0.01 minutes to 20 minutes; wherein said second heating zone is configured to simultaneously heat the top and bottom sides of said formed sheet at a second top heating temperature (Tₜ₂) and a second bottom heating temperature (T_{b2}) for a second heating duration of from 0.01 minutes to 20 minutes; and wherein T_{b1}>Tₜ₁; T_{b1}>T_{b2}; and Tₜ₁<Tₜ₂.

In some embodiments, Tₜ₁ ranges from 80° C. to 150° C., preferably from 80° C. to 140° C.; wherein T_{b1} ranges from 90° C. to 170° C., preferably from 100° C. to 160° C.; wherein Tₜ₂ ranges from 110° C. to 190° C., preferably from 120° C. to 180° C.; and wherein T_{b2} ranges from 70° C. to 150° C., preferably from 70° C. to 120° C.; and wherein T_{b2}<Tₜ₂.

In some embodiments, said multiple heating zones further comprises a third heating zone and wherein said conveying belt is configured to pass through said third heating zone; wherein said third heating zone is configured to simultaneously heat the top and bottom sides of said formed sheet at a third top heating temperature (Tₜ₃) and a third bottom heating temperature (T_{b3}) for a third heating duration of from 0.01 minutes to 20 minutes.

In some embodiments, T_{b2}≥T_{b3}; Tₜ₂≤Tₜ₃; and T_{b3}≤Tₜ₃ when said third heating zone is located downstream of said second heating zone.

In some embodiments, T_{b1}≥T_{b3}≥T_{b2}; Tₜ₁≤Tₜ₃≤Tₜ₂ when said third heating zone is located downstream of said first heating zone and upstream of said second heating zone.

In some embodiments, T_{b3}≥T_{b1}; Tₜ₃≤Tₜ₁; and T_{b3}≥Tₜ₃ when said third heating zone is located upstream of said first heating zone.

In some embodiments, Tₜ₃ ranges from 90° C. to 200° C.; and wherein T_{b3} ranges from 70° C. to 180° C.

In some embodiments, said first heating duration is from 0.1 minutes to 10 minutes, preferably from 0.15 minutes to 8 minutes; and/or said second heating duration is from 0.1 minutes to 10 minutes, preferably from 0.15 minutes to 8 minutes; and/or said third heating duration is from 0.1 minutes to 10 minutes, preferably from 0.15 minutes to 8 minutes; and/or the total heating duration in said multiple heating zones is from 0.3 minutes to 30 minutes, preferably from 0.5 minutes to 20 minutes, more preferably from 0.6 minutes to 15 minutes.

In a preferred embodiment, the porous sheet according to the present disclosure and/or the dissolvable solid article according to the present disclosure is characterized by:
a Percent Open Cell Content of from 85% to 100%, preferably from 90% to 100%; and/or
an Overall Average Pore Size of from 150 μm to 1000 μm, preferably from 200 μm to 600 μm; and/or
an Average Cell Wall Thickness of from 5 μm to 200 μm, preferably from 10 μm to 100 μm, more preferably from 10 μm to 80 μm; and/or
a final moisture content of from 0.5% to 25%, preferably from 1% to 20%, more preferably from 3% to 10%, by weight of the solid sheet article; and/or.
a thickness of from 0.6 mm to 3.5 mm, preferably from 0.7 mm to 3 mm, more preferably from 0.8 mm to 2 mm, most preferably from 1 mm to 2 mm; and/or
a basis weight of from about 50 grams/m² to about 500 grams/m², preferably from about 150 grams/m² to about 450 grams/m², more preferably from about 250 grams/m² to about 400 grams/m²; and/or
a density of from 0.05 grams/cm³ to 0.5 grams/cm³, preferably from 0.06 grams/cm³ to 0.4 grams/cm³, more preferably from 0.07 grams/cm³ to 0.2 grams/cm³, most preferably from 0.08 grams/cm³ to 0.15 grams/cm³; and/or
a Specific Surface Area of from 0.03 m²/g to 0.25 m²/g, preferably from 0.04 m²/g to 0.22 m²/g, more preferably from 0.05 m²/g to 0.2 m²/g, most preferably from 0.1 m²/g to 0.18 m²/g.

### III. FORMULATIONS OF POROUS SHEETS

### 1. WATER-SOLUBLE POLYMER

The flexible, porous, dissolvable solid sheet of the present invention may be formed by a wet pre-mixture that comprises a water-soluble polymer and a first surfactant. Such a water-soluble polymer may function in the resulting solid sheet as a film-former, a structurant as well as a carrier for other active ingredients (e.g., surfactants, emulsifiers, builders, chelants, perfumes, colorants, and the like).

Preferably, the wet pre-mixture may comprise from about 3% to about 20% by weight of the pre-mixture of water-soluble polymer, in one embodiment from about 5% to about 15% by weight of the pre-mixture of water-soluble polymer, in one embodiment from about 7% to about 10% by weight of the pre-mixture of water-soluble polymer.

After drying, it is preferred that the water-soluble polymer is present in the flexible, porous, dissolvable solid sheet of the present invention in an amount ranging from about 5% to about 60%, preferably from about 7% to about 50%, more preferably from about 9% to about 40%, most preferably from about 10% to about 30%, for example 10%, 12%, 15%, 18%, 20%, 25%, 30% or any ranges therebetween, by total weight of the solid sheet. In a particularly preferred embodiment of the present invention, the total amount of water-soluble polymer(s) present in the flexible, porous, dissolvable solid sheet of the present invention is no more than 25% by total weight of such sheet.

Water-soluble polymers suitable for the practice of the present invention may be selected those with weight average molecular weights ranging from about 5,000 to about 400,000 Daltons, preferably from about 10,000 to about 300,000 Daltons, more preferably from about 15,000 to about 200,000 Daltons, most preferably from about 20,000 to about 150,000 Daltons. The weight average molecular weight is computed by summing the average molecular weights of each polymer raw material multiplied by their respective relative weight percentages by weight of the total weight of polymers present within the porous solid sheet. The weight average molecular weight of the water-soluble polymer used herein may impact the viscosity of the wet pre-mixture, which may in turn influence the bubble number and size during the aeration step as well as the pore expansion/opening results during the drying step. Further, the weight average molecular weight of the water-soluble polymer may affect the overall film-forming properties of the wet pre-mixture and its compatibility/incompatibility with certain surfactants.

The water-soluble polymers of the present invention may include, but are not limited to, synthetic polymers including polyvinyl alcohols, polyvinylpyrrolidones, polyalkylene oxides, polyacrylates, caprolactams, polymethacrylates, polymethylmethacrylates, polyacrylamides, polymethylacrylamides, polydimethylacrylamides, polyethylene glycol monomethacrylates, copolymers of acrylic acid and methyl acrylate, polyurethanes, polycarboxylic acids, polyvinyl acetates, polyesters, polyamides, polyamines, polyethyleneimines, maleic/(acrylate or methacrylate) copolymers, copolymers of methylvinyl ether and of maleic anhydride, copolymers of vinyl acetate and crotonic acid, copolymers of vinylpyrrolidone and of vinyl acetate, copolymers of vinylpyrrolidone and of caprolactam, vinyl pyrollidone/vinyl acetate copolymers, copolymers of anionic, cationic and amphoteric monomers, and combinations thereof.

The water-soluble polymers of the present invention may also be selected from naturally sourced polymers including those of plant origin examples of which include karaya gum, tragacanth gum, gum Arabic, acemannan, konjac mannan, acacia gum, gum ghatti, whey protein isolate, and soy protein isolate; seed extracts including guar gum, locust bean gum, quince seed, and psyllium seed; seaweed extracts such as Carrageenan, alginates, and agar; fruit extracts (pectins); those of microbial origin including xanthan gum, gellan gum, pullulan, hyaluronic acid, chondroitin sulfate, and dextran; and those of animal origin including cascin, gelatin, keratin, keratin hydrolysates, sulfonic keratins, albumin, collagen, glutelin, glucagons, gluten, zein, and shellac.

Modified natural polymers can also be used as water-soluble polymers in the present invention. Suitable modified natural polymers include, but are not limited to, cellulose derivatives such as hydroxypropylmethylcellulose, hydroxymethylcellulose, hydroxyethylcellulose, methylcellulose, hydroxypropylcellulose, ethylcellulose, carboxymethylcellulose, cellulose acetate phthalate, nitrocellulose and other cellulose ethers/esters; and guar derivatives such as hydroxypropyl guar.

Preferred water-soluble polymers of the present invention include polyvinyl alcohols, polyvinylpyrrolidones, polyalkylene oxides, starch and starch derivatives, pullulan, gelatin, hydroxypropylmethylcelluloses, methycelluloses, and carboxymethycelluloses. More preferred water-soluble polymers of the present invention include polyvinyl alcohols, and hydroxypropylmethylcelluloses.

Most preferred water-soluble polymers of the present invention are polyvinyl alcohols characterized by a degree of hydrolysis ranging from about 40% to about 100%, preferably from about 50% to about 95%, more preferably from about 65% to about 92%, most preferably from about 70% to about 90%. Commercially available polyvinyl alcohols include those from Celanese Corporation (Texas, USA) under the CELVOL trade name including, but not limited to, CELVOL 523, CELVOL 530, CELVOL 540, CELVOL 518, CELVOL 513, CELVOL 508, CELVOL 504;those from Kuraray Europe GmbH (Frankfurt, Germany) under the Mowiol® and POVAL™ trade names; and PVA 1788 (also referred to as PVA BP17) commercially available from various suppliers including Lubon Vinylon Co. (Nanjing, China); and combinations thereof. In a particularly preferred embodiment of the present invention, the flexible, porous, dissolvable solid sheet comprises from about 10% to about 25%, more preferably from about 15% to about 23%, by total weight of such sheet, of a polyvinyl alcohol having a weight average molecular weight ranging from 80,000 to about 150,000 Daltons and a degree of hydrolysis ranging from about 80% to about 90%.

### 2. SURFACTANTS

In addition to the water-soluble polymer described hereinabove, the solid sheet of the present invention comprises a surfactant. The surfactant may function as emulsifying agents during the aeration process to create a sufficient amount of stable bubbles for forming the desired OCF structure of the present invention. Further, the surfactant may function as active ingredients for delivering a desired cleansing benefit.

In a preferred embodiment of the present invention, the solid sheet comprises a surfactant selected from the group consisting of anionic surfactants, nonionic surfactants, cationic surfactants, zwitterionic surfactants, amphoteric surfactants, polymeric surfactants and any combinations thereof. Depending on the desired application of such solid sheet and the desired consumer benefit to be achieved, different surfactants can be selected. One benefit of the present invention is that the OCF structures of the solid sheet allow for incorporation of a high surfactant content while still providing fast dissolution. Consequently, highly concentrated cleansing compositions can be formulated into the solid sheets of the present invention to provide a new and superior cleansing experience to the consumers.

The surfactant as used herein may include both surfactants from the conventional sense (i.e., those providing a consumer-noticeable lathering effect) and emulsifiers (i.e., those that do not provide any lathering performance but are intended primarily as a process aid in making a stable foam structure). Examples of emulsifiers for use as a surfactant component herein include mono- and di-glycerides, fatty alcohols, polyglycerol esters, propylene glycol esters, sorbitan esters and other emulsifiers known or otherwise commonly used to stabilize air interfaces.

The total amount of the surfactant present in the solid sheet of the present invention may range widely from about 5% to about 95%, preferably from about 30% to about 90%, preferably from about 40% to about 80%, more preferably from about 50% to about 70%, e.g. 20%, 30%, 40%, 50%, 60%, 70%, 80% or any ranges therebetween, by total weight of the solid sheet. Correspondingly, the wet pre-mixture may comprise from about 1% to about 50% by weight of the wet pre-mixture of surfactant(s), in one embodiment from about 2% to about 40% by weight of the wet pre-mixture of surfactant(s), in one embodiment from about 10% to about 35% by weight of the wet pre-mixture of surfactant(s), in one embodiment from about 15% to about 30% by weight of the wet pre-mixture of surfactant(s).

Non-limiting examples of anionic surfactants suitable for use herein include alkyl and alkyl ether sulfates, sulfated monoglycerides, sulfonated olefins, alkyl aryl sulfonates, primary or secondary alkane sulfonates, alkyl sulfosuccinates, acyl taurates, acyl isethionates, alkyl glycerylether sulfonate, sulfonated methyl esters, sulfonated fatty acids, alkyl phosphates, acyl glutamates, acyl sarcosinates, alkyl sulfoacetates, acylated peptides, alkyl ether carboxylates, acyl lactylates, anionic fluorosurfactants, sodium lauroyl glutamate, and combinations thereof.

One category of anionic surfactants particularly suitable for practice of the present invention include C₆-C₂₀ linear alkylbenzene sulphonate (LAS) surfactant. LAS surfactants are well known in the art and can be readily obtained by sulfonating commercially available linear alkylbenzenes. Exemplary C₁₀-C₂₀ linear alkylbenzene sulfonates that can be used in the present invention include alkali metal, alkaline earth metal or ammonium salts of C₁₀-C₂₀ linear alkylbenzene sulfonic acids, and preferably the sodium, potassium, magnesium and/or ammonium salts of C₁₁-C₁₈ or C₁-C₁₄ linear alkylbenzene sulfonic acids. More preferred are the sodium or potassium salts of C₁₂ and/or C₁₄ linear alkylbenzene sulfonic acids, and most preferred is the sodium salt of C₁₂ and/or C₁₄ linear alkylbenzene sulfonic acid, i.e., sodium dodecylbenzene sulfonate or sodium tetradecylbenzene sulfonate.

LAS provides superior cleaning benefit and is especially suitable for use in laundry detergent applications. It has been a surprising and unexpected discovery of the present invention that when polyvinyl alcohol having a higher weight average molecular weight (e.g., from about 50,000 to about 400,000 Daltons, preferably from about 60,000 to about 300,000 Daltons, more preferably from about 70,000 to about 200,000 Daltons, most preferably from about 80,000 to about 150,000 Daltons) is used as the film-former and carrier, LAS can be used as a major surfactant, i.e., present in an amount that is more than 50% by weight of the total surfactant content in the solid sheet, without adversely affecting the film-forming performance and stability of the overall composition. Correspondingly, in a particular embodiment of the present invention, LAS is used as the major surfactant in the solid sheet. If present, the amount of LAS in the solid sheet of the present invention may range from about 10% to about 70%, preferably from about 20% to about 65%, more preferably from about 40% to about 60%, by total weight of the solid sheet.

Another category of anionic surfactants suitable for practice of the present invention include sodium trideceth sulfates (STS) having a weight average degree of alkoxylation ranging from about 0.5 to about 5, preferably from about 0.8 to about 4, more preferably from about 1 to about 3, most preferably from about 1.5 to about 2.5. Trideceth is a 13-carbon branched alkoxylated hydrocarbon comprising, in one embodiment, an average of at least 1 methyl branch per molecule. STS used by the present invention may be include ST(EOxPOy)S, while EOx refers to repeating ethylene oxide units with a repeating number x ranging from 0 to 5, preferably from 1 to 4, more preferably from 1 to 3, and while POy refers to repeating propylene oxide units with a repeating number y ranging from 0 to 5, preferably from 0 to 4, more preferably from 0 to 2. It is understood that a material such as ST2S with a weight average degree of ethoxylation of about 2, for example, may comprise a significant amount of molecules which have no ethoxylate, 1 mole ethoxylate, 3 mole ethoxylate, and so on, while the distribution of ethoxylation can be broad, narrow or truncated, which still results in an overall weight average degree of ethoxylation of about 2. STS is particularly suitable for personal cleansing applications, and it has been a surprising and unexpected discovery of the present invention that when polyvinyl alcohol having a higher weight average molecular weight (e.g., from about 50,000 to about 400,000 Daltons, preferably from about 60,000 to about 300,000 Daltons, more preferably from about 70,000 to about 200,000 Daltons, most preferably from about 80,000 to about 150,000 Daltons) is used as the film-former and carrier, STS can be used as a major surfactant, i.e., present in an amount that is more than 50% by weight of the total surfactant content in the solid sheet, without adversely affecting the film-forming performance and stability of the overall composition. Correspondingly, in a particular embodiment of the present invention, STS is used as the major surfactant in the solid sheet. If present, the amount of STS in the solid sheet of the present invention may range from about 10% to about 70%, preferably from about 20% to about 65%, more preferably from about 40% to about 60%, by total weight of the solid sheet.

Another category of anionic surfactants suitable for practice of the present invention include alkyl sulfates. These materials have the respective formulae ROSO3M, wherein R is alkyl or alkenyl of from about 6 to about 20 carbon atoms, x is 1 to 10, and M is a water-soluble cation such as ammonium, sodium, potassium and triethanolamine. Preferably, R has from about 6 to about 18, preferably from about 8 to about 16, more preferably from about 10 to about 14, carbon atoms. Previously, unalkoxylated C₆-C₂₀ linear or branched alkyl sulfates (AS) have been considered the preferred surfactants in dissolvable solid sheets, especially as the major surfactant therein, due to its compatibility with low molecular weight polyvinyl alcohols (e.g., those with a weight average molecular weight of no more than 50,000 Daltons) in film-forming performance and storage stability. However, it has been a surprising and unexpected discovery of the present invention that when polyvinyl alcohol having a higher weight average molecular weight (e.g., from about 50,000 to about 400,000 Daltons, preferably from about 60,000 to about 300,000 Daltons, more preferably from about 70,000 to about 200,000 Daltons, most preferably from about 80,000 to about 150,000 Daltons) is used as the film-former and carrier, other surfactants, such as LAS and/or STS, can be used as the major surfactant in the solid sheet, without adversely affecting the film-forming performance and stability of the overall composition. Therefore, in a particularly preferred embodiment of the present invention, it is desirable to provide a solid sheet with no more than about 20%, preferably from 0% to about 10%, more preferably from 0% to about 5%, most preferably from 0% to about 1%, by weight of the solid sheet, of AS.

Another category of anionic surfactants suitable for practice of the present invention include C₆-C₂₀ linear or branched alkylalkoxy sulfates (AAS). Among this category, linear or branched alkylethoxy sulfates (AES) having the respective formulae RO(C₂H₄O)_{X}SO₃M are particularly preferred, wherein R is alkyl or alkenyl of from about 6 to about 20 carbon atoms, x is 1 to 10, and M is a water-soluble cation such as ammonium, sodium, potassium and triethanolamine. Preferably, R has from about 6 to about 18, preferably from about 8 to about 16, more preferably from about 10 to about 14, carbon atoms.

Nonionic surfactants that can be included into the solid sheet of the present invention may be any conventional nonionic surfactants, including but not limited to: alkyl alkoxylated alcohols, alkyl alkoxylated phenols, alkyl polysaccharides (especially alkyl glucosides and alkyl polyglucosides), polyhydroxy fatty acid amides, alkoxylated fatty acid esters, sucrose esters, sorbitan esters and alkoxylated derivatives of sorbitan esters, amine oxides, and the like. Preferred nonionic surfactants are those of the formula R¹(OCH₄)ₙOH, wherein R¹ is a C₈-C₁₈ alkyl group or alkyl phenyl group, and n is from about 1 to about 80. Particularly preferred are C₈-C₁₈ alkyl ethoxylated alcohols having a weight average degree of ethoxylation from about 1 to about 20, preferably from about 5 to about 15, more preferably from about 7 to about 10, such as NEODOL® nonionic surfactants commercially available from Shell. Other non-limiting examples of nonionic surfactants useful herein include: C₆-C₁₂ alkyl phenol alkoxylates where the alkoxylate units may be ethyleneoxy units, propyleneoxy units, or a mixture thereof; C₁₂-Cis alcohol and C₆-C₁₂ alkyl phenol condensates with ethylene oxide/propylene oxide block polymers such as Pluronic® from BASF; C₁₄-C₂₂ mid-chain branched alcohols (BA); C₁₄-C₂₂ mid-chain branched alkyl alkoxylates, BAEₓ, wherein x is from 1 to 30; alkyl polysaccharides, specifically alkyl polyglycosides; Polyhydroxy fatty acid amides; and ether capped poly(oxyalkylated) alcohol surfactants. Suitable nonionic surfactants also include those sold under the tradename Lutensol® from BASF.

The most preferred nonionic surfactants for practice of the present invention include C₆-C₂₀ linear or branched alkylalkoxylated alcohols (AA) having a weight average degree of alkoxylation ranging from 5 to 15, more preferably C₁₂-C₁₄ linear ethoxylated alcohols having a weight average degree of alkoxylation ranging from 7 to 9. If present, the amount of AA-type nonionic surfactant(s) in the solid sheet of the present invention may range from about 2% to about 40%, preferably from about 5% to about 30%, more preferably from about 8% to about 12%, by total weight of the solid sheet.

Amphoteric surfactants suitable for use in the solid sheet of the present invention includes those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, sodium lauryl sarcosinate, N-alkyltaurines such as the one prepared by reacting dodecylamine with sodium isethionate, and N-higher alkyl aspartic acids.

Zwitterionic surfactants suitable include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Such suitable zwitterionic surfactants can be represented by the formula:

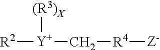

wherein R² contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing about 1 to about 3 carbon atoms; X is 1 when Y is a sulfur atom, and 2 when Y is a nitrogen or phosphorus atom; R⁴ is an alkylene or hydroxyalkylene of from about 1 to about 4 carbon atoms and Z is a radical selected from the group consisting of carboxylate, sulfonate, sulfate, phosphonate, and phosphate groups.

Cationic surfactants can also be utilized in the present invention, especially in fabric softener and hair conditioner products. When used in making products that contain cationic surfactants as the major surfactants, it is preferred that such cationic surfactants are present in an amount ranging from about 2% to about 30%, preferably from about 3% to about 20%, more preferably from about 5% to about 15% by total weight of the solid sheet. Cationic surfactants may include DEQA compounds, which encompass a description of diamido actives as well as actives with mixed amido and ester linkages. Preferred DEQA compounds are typically made by reacting alkanolamines such as MDEA (methyldiethanolamine) and TEA (triethanolamine) with fatty acids. Some materials that typically result from such reactions include N,N-di(acyl-oxyethyl)-N,N-dimethylammonium chloride or N,N-di(acyl-oxyethyl)-N,N-methylhydroxyethylammonium methylsulfate wherein the acyl group is derived from animal fats, unsaturated, and polyunsaturated, fatty acids.

Suitable polymeric surfactants for use in the personal care compositions of the present invention include, but are not limited to, block copolymers of ethylene oxide and fatty alkyl residues, block copolymers of ethylene oxide and propylene oxide, hydrophobically modified polyacrylates, hydrophobically modified celluloses, silicone polyethers, silicone copolyol esters, diquaternary polydimethylsiloxanes, and co-modified amino/polyether silicones.

In a preferred embodiment, the surfactant may be selected from the group consisting of a C₆-C₂₀ linear alkylbenzene sulfonate (LAS), a C₆-C₂₀ linear or branched alkylalkoxy sulfates (AAS) having a weight average degree of alkoxylation ranging from 0.5 to 10, a C₆-C₂₀ linear or branched alkylalkoxylated alcohols (AA) having a weight average degree of alkoxylation ranging from 5 to 15, a C₆-C₂₀ linear or branched alkyl sulfates (AS) and any combinations thereof.

### 3. PLASTICIZERS

In a preferred embodiment of the present invention, the flexible, porous, dissolvable solid sheet of the present invention may further comprise a plasticizer, preferably in the amount ranging from about 0.1% to about 25%, preferably from about 0.5% to about 20%, more preferably from about 1% to about 15%, most preferably from 2% to 12%, by total weight of the solid sheet. Correspondingly, the wet pre-mixture used for forming such solid sheet may comprise from about 0.02% to about 20% of a plasticizer by weight of the wet pre-mixture, in one embodiment from about 0.1% to about 10% of a plasticizer by weight of the wet pre-mixture, in one embodiment from about 0.5% to about 5% of a plasticizer by weight of the wet pre-mixture.

Suitable plasticizers for use in the present invention include, for example, polyols, copolyols, polycarboxylic acids, polyesters, dimethicone copolyols, and the like.

Examples of useful polyols include, but are not limited to: glycerin, diglycerin, ethylene glycol, polyethylene glycol (especially 200-600), propylene glycol, butylene glycol, pentylene glycol, glycerol derivatives (such as propoxylated glycerol), glycidol, cyclohexane dimethanol, hexanediol, 2,2,4-trimethylpentane-1,3-diol, pentacrythritol, urea, sugar alcohols (such as sorbitol, mannitol, lactitol, xylitol, maltitol, and other mono-and polyhydric alcohols), mono-, di- and oligo-saccharides (such as fructose, glucose, sucrose, maltose, lactose, high fructose corn syrup solids, and dextrins), ascorbic acid, sorbates, ethylene bisformamide, amino acids, and the like.

Examples of polycarboxylic acids include, but are not limited to citric acid, maleic acid, succinic acid, polyacrylic acid, and polymaleic acid.

Examples of suitable polyesters include, but are not limited to, glycerol triacetate, acetylated-monoglyceride, diethyl phthalate, triethyl citrate, tributyl citrate, acetyl triethyl citrate, acetyl tributyl citrate.

Examples of suitable dimethicone copolyols include, but are not limited to, PEG-12 dimethicone, PEG/PPG-18/18 dimethicone, and PPG-12 dimethicone.

Particularly preferred examples of plasticizers include glycerin, ethylene glycol, polyethylene glycol, propylene glycol, and mixtures thereof. Most preferred plasticizer is glycerin.

### 4. ADDITIONAL INGREDIENTS

In addition to the above-described ingredients, e.g., the water-soluble polymer, the surfactant(s) and the plasticizer, the solid sheet of the present invention may comprise one or more additional ingredients, depending on its intended application. Such one or more additional ingredients may be selected from the group consisting of fabric care actives, dishwashing actives, hard surface cleaning actives, beauty and/or skin care actives, personal cleansing actives, hair care actives, oral care actives, feminine care actives, baby care actives, a bittering agent and any combinations thereof. In a preferred embodiment, the solid sheet of the present invention may comprise a bittering agent.

The solid sheet of the present invention may further comprise other optional ingredients that are known for use or otherwise useful in compositions, provided that such optional materials are compatible with the selected essential materials described herein, or do not otherwise unduly impair product performance.

Non-limiting examples of product type embodiments that can be formed by the solid sheet of the present invention include laundry detergent products, fabric softening products, hand cleansing products, hair shampoo or other hair treatment products, body cleansing products, shaving preparation products, dish cleaning products, personal care substrates containing pharmaceutical or other skin care actives, moisturizing products, sunscreen products, beauty or skin care products, deodorizing products, oral care products, feminine cleansing products, baby care products, fragrance-containing products, and so forth.

### IV. FORMULATIONS OF LOADING COMPOSITION

The loading composition in a form of paste according to the present disclosure may comprise a non-aqueous liquid carrier, solid particles and a polyalkylene polymer. The loading composition in a form of powders according to the present disclosure may comprise solid particles.

### 1. NON-AQUEOUS LIQUID CARRIER

The loading composition may comprise from 1% to 99%, preferably from 5% to 70%, more preferably from 20% to 50%, for example 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95% or any ranges therebetween, of the non-aqueous liquid carrier by total weight of the loading composition.

The non-aqueous liquid carrier may be selected from the group consisting of polyethylene glycol, polypropylene glycol, silicone, fatty acid, perfume oil, a non-ionic surfactant, an organic solvent and any combinations thereof. Preferably, the non-aqueous liquid carrier may comprise a non-ionic surfactant. The non-ionic surfactant may be any appropriate non-ionic surfactant as listed hereinbefore. In a more preferred embodiment, the non-ionic surfactant may comprise a C₆-C₂₀ linear or branched alkylalkoxylated alcohols (AA) having a weight average degree of alkoxylation ranging from 5 to 15, preferably C₁₂-C₁₄ linear ethoxylated alcohols having a weight average degree of alkoxylation ranging from 7 to 9. In another embodiment, the non-aqueous liquid carrier may comprise polyethylene glycol having a weight average molecular weight of less than 1000, less than 800, or less than 600.

### 2. POLYALKYLENE POLYMER

The loading composition may comprise from 0.5% to 80%, preferably from 0.5% to 50%, more preferably from 0.8% to 30%, most preferably from 1% to 20%, for example 0.5%, 0.6%, 0.7%, 0.8%, 0.9%, 1.0%, 1.5%, 2.0%, 2.5%, 3.0%, 3.5%, 4.0%, 4.5%, 5.0%, 6.0%, 7.0%, 8.0%, 9.0%, 10%, 15%, 20%, 30%, or any ranges therebetween, of the polymer by total weight of the loading composition.

In some embodiments, the polyalkylene polymer is selected from a group consisting of polyalkylene imine polymer, polyalkylene oxide polymer and any combinations thereof. Preferably said polyalkylene polymer is a polyalkylene graft copolymer comprising a) polyalkylene oxide component as a graft base, and b) polyvinyl ester component as side chains, and/or c) polyvinylpyrrolidone as side chains. More preferably said polyalkylene polymer is a polyalkylene graft copolymer comprising a) polyalkylene oxide component as a graft base, which has a number average molecular weight of from 1000 to 20,000 Daltons and is based on ethylene oxide, propylene oxide, butylene oxide or mixtures thereof, b) polyvinyl ester component as side chains, which is derived from a saturated monocarboxylic acid containing from 1 to 6 carbon atoms and/or a methyl or ethyl ester of acrylic or methacrylic acid, and c) polyvinylpyrrolidone as side chains, wherein the weight ratio of (a):(c) is from 1:0.1 to 1:2, preferably from 1:0.1 to 1:1, more preferably from 1:0.3 to 1:1, and wherein the amount, by weight, of (a) is greater than the amount of (b).

### Polyalkylene Imine Polymers

The polyalkylene polymer may be a polyalkylene imine polymer which comprising a core structure and a plurality of alkoxylate groups. The core structure may comprise either i) a polyalkylenimine structure comprising, in condensed form, repeating units of formulae (I), (II), (III) and (IV):

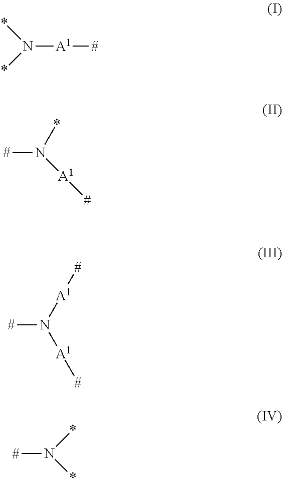

wherein #in each case denotes one-half of a bond between a nitrogen atom and the free binding position of a group A¹ of two adjacent repeating units of formulae (I), (II), (III) or (IV); * in each case denotes one-half of a bond to one of the alkoxylate groups; and A¹ is independently selected from linear or branched C₂-C₆-alkylene; wherein the polyalkylenimine structure consists of 1 repeating unit of formula (I), x repeating units of formula (II), y repeating units of formula (III) and y+1 repeating units of formula (IV), wherein x and y in each case have a value in the range of from 0 to about 150; where the average weight average molecular weight, Mw, of the polyalkylenimine core structure is a value in the range of from about 60 to about 10,000 g/mol; or ii) a polyalkanolamine structure of the condensation products of at least one compound selected from N-(hydroxyalkyl) amines of formulae (I.a) and/or (I.b),

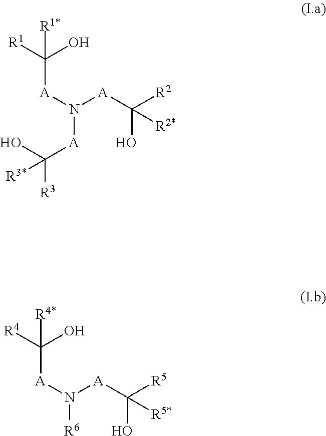

wherein A are independently selected from C₁-C₆-alkylene; R¹, R¹*, R², R²*, R³, R³*, R⁴, R⁴*, R⁵ and R⁵* are independently selected from hydrogen, alkyl, cycloalkyl or aryl, wherein the last three mentioned radicals may be optionally substituted; and R⁶ is selected from hydrogen, alkyl, cycloalkyl or aryl, wherein the last three mentioned radicals may be optionally substituted. The the plurality of alkylenoxy groups are independently selected from alkylenoxy units of the formula (V)

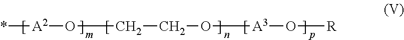

wherein: * in each case denotes one-half of a bond to the nitrogen atom of the repeating unit of formula (I), (II) or (IV); A² is in each case independently selected from 1,2-propylene, 1,2-butylene and 1,2-isobutylene; A³ is 1,2-propylene; R is in each case independently selected from hydrogen and C₁-C₄-alkyl; m has an average value in the range of from 0 to about 2; n has an average value in the range of from about 20 to about 50; and p has an average value in the range of from about 10 to about 50.

### Polyalkylene Oxide Polymers

The polyalkylene polymer may be a polyalkylene oxide polymer. In a particular embodiment, the polyalkylene oxide polymer is a graft polymer based on water-soluble polyalkylene oxides (A) as a graft base and side chains formed by polymerization of a vinyl ester component (B), where the polymer has a mean molar mass (Mw) of from 3000 to 100,000 and where the polymer comprises (A) from 15% to 70%, preferably from 20% to 70%, more preferably from 25% to 60%, by weight of a water-soluble polyalkylene oxide as a graft base and (B) side chains formed by free-radical polymerization of from 30 to 85%, preferably from 30% to 80%, more preferably from 40% to 75%, by weight of a vinyl ester component composed of (B1) from 70 to 100% by weight of vinyl acetate and/or vinyl propionate and (B2) from 0 to 30% by weight of a further ethylenically unsaturated monomer, where the polymer has a full width at half maximum of the polarity distribution between 0.35 and 1.0:

The graft copolymer comprises and/or is obtainable by grafting (a) a polyalkylene oxide which has a number average molecular weight of from 1000 to 20000 Da, or to 15000, or to 12000 Da, or to 10000 Da and is based on ethylene oxide, propylene oxide, or butylene oxide, preferably based on ethylene oxide, with (b) a vinyl ester component.

Water-soluble polyalkylene oxides suitable for forming the graft base (A) are in principle all polymers based on C2-C4-alkylene oxides which comprise at least 50% by weight, preferably at least 60% by weight, more preferably at least 75% by weight of ethylene oxide in copolymerized form.

The polyalkylene oxides (A) preferably have a low polydispersity M_{w}/Mn. Their polydispersity is preferably less than 1.5.

The polyalkylene oxides (A) may be the corresponding polyalkylene glycols in free form, i.e. with OH end groups, but they may also be capped at one or both end groups. Suitable end groups are, for example, C₁-C₂₅-alkyl, phenyl and C₁-C₁₄-alkylphenyl groups. Specific examples of particularly suitable polyalkylene oxides (A) include:
(A1) polyethylene glycols which may be capped at one or both end groups, especially by C₁-C₂₅-alkyl groups, but are preferably not etherified, and have mean molar masses Mn of preferably from 1500 to 20,000, more preferably from 2500 to 15,000;
(A2) copolymers of ethylene oxide and propylene oxide and/or butylene oxide with an ethylene oxide content of at least 50% by weight, which may likewise be capped at one or both end groups, especially by C₁-C₂₅-alkyl groups, but are preferably not etherified, and have mean molar masses Mₙ of preferably from 1500 to 20,000, more preferably from 2500 to 15,000;
(A3) chain-extended products having mean molar masses of in particular from 2500 to 20,000, which are obtainable by reacting polyethylene glycols (A1) having mean molar masses Mₙ of from 200 to 5000 or copolymers (A2) having mean molar masses Mₙ of from 200 to 5000 with C₂-C₁₂-dicarboxylic acids or-dicarboxylic esters or C₆-C₁₈-diisocyanates.

Preferred graft bases (A) are the polyethylene glycols (A1).

The polyalkylene oxide backbone of the graft copolymer of the present invention, which is also referred to herein as the graft base, may comprise repeated units of C₂-C₁₀, preferably C₂-C₆, and more preferably C₂-C₄, alkylene oxides. For example, the polyalkylene oxide backbone may be: a polyethylene oxide (PEO) backbone; a polypropylene oxide (PPO) backbone; a polybutylene oxide (PBO) backbone; a polymeric backbone that is a linear block copolymer of PEO, PPO, and/or PBO; and combinations thereof. Preferably, the polyalkylene oxide backbone is a PEO backbone. Such a polyalkylene oxide backbone preferably has a number average molecular weight (Mₙ) from about 1,000 to about 20,000 g/mol, preferably from about 2,000 to about 15,000 g/mol, more preferably from about 3,000 to about 13,000 g/mol, and most preferably from about 5,000 to about 10,000 g/mol.

The one or more side chains of the graft copolymers of the present invention are formed by polymerizations of a vinyl ester component in the presence of the graft base. Suitable vinyl ester components may be selected from C₂-C₁₀ vinyl esters, preferably C₂-C₆ vinyl esters, and more preferably C₂-C₄ vinyl carboxylates. For example, the one or more side chains may be selected from the group consisting of polyvinyl acetate, polyvinyl propionate, polyvinyl butyrate, and combinations thereof, while polyvinyl acetate is preferred. The side chains may also be formed by copolymerizing vinyl acetate and/or vinyl propionate with a further ethylenically unsaturated monomer (e.g., methyl acrylate, ethyl acrylate, and n-butyl acrylate). The fraction of such further ethylenically unsaturated monomer in the total content of the vinyl ester component may be up to 30% by weight. The polyvinyl ester side chains may further be partially saponified, for example, to an extent of up to 15%.

Such side chains may be present in an amount ranging from about 30% to about 85%, preferably from about 40% to about 75%, by total weight of the graft copolymer.

The graft copolymers of the present invention may have an overall weight average molecular weight (M_{w}) of from about 3,000 to about 100,000, preferably from about 10,000 to about 50,000, and more preferably from about 20,000 to about 40,000.

The graft copolymers of the present invention may further feature a narrow molar mass distribution represented by a polydispersity M_{w}/Mₙ of generally ≤about 3, preferably ≤about 2.8, more preferably ≤about 2.5, and even more preferably ≤about 2.3. Most preferably, their polydispersity M_{w}/Mₙ is in the range from about 1.5 to about 2.2.

The vinyl ester component (B) may consist advantageously of (B1) vinyl acetate or vinyl propionate or of mixtures of vinyl acetate and vinyl propionate, particular preference being given to vinyl acetate as the vinyl ester component (B).

However, the side chains of the graft polymer can also be formed by copolymerizing vinyl acetate and/or vinyl propionate (B1) and a further ethylenically unsaturated monomer (B2). The fraction of monomer (B2) in the vinyl ester component (B) may be up to 30% by weight, which corresponds to a content in the graft polymer of (B2) of 24% by weight. Suitable comonomers (B2) are, for example, monoethylenically unsaturated carboxylic acids and dicarboxylic acids and their derivatives, such as esters, amides and anhydrides, and styrene. It is of course also possible to use mixtures of different comonomers. Specific examples include: (meth) acrylic acid, C₁-C₁₂-alkyl and hydroxy-C₂-C₁₂-alkyl esters of (meth)acrylic acid, (meth)acrylamide, N-C₁-C₁₂-alkyl(meth)acrylamide, N,N-di(C₁-C₆-alkyl)(meth)acrylamide, maleic acid, maleic anhydride and mono(C₁-C₁₂-alkyl)esters of maleic acid.

In a particularly embodiment, the polyalkylene oxide graft copolymer comprises: (a) polyalkylene oxide which has a number average molecular weight of from 1000 to 20,000 Daltons and is based on ethylene oxide, propylene oxide, and/or butylene oxide, (b) polyvinyl ester component as side chains, and (c) polyvinylpyrrolidone as side chains. Particularly, the polyvinyl ester component is derived from a saturated monocarboxylic acid containing from 1 to 6 carbon atoms.

Suitable polyalkylene oxides may be based on homopolymers or copolymers, with homopolymers being preferred. Suitable polyalkylene oxides may be based on homopolymers of ethylene oxide or ethylene oxide copolymers having an ethylene oxide content of from 40 mol % to 99 mol %. Suitable comonomers for such copolymers may include propylene oxide, n-butylene oxide, and/or isobutylene oxide. Suitable copolymers may include copolymers of ethylene oxide and propylene oxide, copolymers of ethylene oxide and butylene oxide, and/or copolymers of ethylene oxide, propylene oxide, and at least one butylene oxide. The copolymers may include an ethylene oxide content of from 40 to 99 mol %, a propylene oxide content of from 1.0 to 60 mol %, and a butylene oxide content of from 1.0 to 30 mol %. The graft base may be linear (straight-chain) or branched, for example a branched homopolymer and/or a branched copolymer.

Branched copolymers may be prepared by addition of ethylene oxide with or without propylene oxides and/or butylene oxides onto polyhydric low molecular weight alcohols, for example trimethylol propane, pentoses, or hexoses.

The alkylene oxide unit may be randomly distributed in the polymer or be present therein as blocks.

The polyalkylene oxides of component (a) may be the corresponding polyalkylene glycols in free form, that is, with OH end groups, or they may be capped at one or both end groups. Suitable end groups may be, for example, C1-C25-alkyl, phenyl, and C1-C14-alkylphenyl groups. The end group may be a Cl-alkyl (e.g., methyl) group. Suitable materials for the graft base may include PEG 1000, PEG 2000, PEG 4000, PEG 6000, PEG 8000, PEG 10000, PEG 12000, and/or PEG 20000, which are polyethylene glycols, and/or MPEG 2000, MPEG 4000, MPEG 6000, MPEG 8000 and MEG 10000 which are monomethoxypolyethylene glycols that are commercially available from BASF under the tradename PLURIOLand/or block copolymers made from ethylene oxide-propylene oxide-ethylene oxide (EO-PO-EO) or from propylene oxide-ethylene oxide-propylene oxide (PO-EO-PO) such as PE 6100, PE 6800 or PE 3100 commercially available from BASF under the tradename PLURONIC.

The polyalkylene oxides may be grafted with N-vinylpyrrolidone as the monomer of component (b). Without wishing to be bound by theory, it is believed that the presence of the N-vinylpyrrolidone (“VP”) monomer in the graft copolymers according to the present disclosure provides water-solubility and good film-forming properties compared to otherwise-similar polymers that do not contain the N-vinylpyrrolidone monomer. The vinyl pyrrolidone repeat unit has amphiphilic character with a polar amide group that can form a dipole, and a non-polar portion with the methylene groups in the backbone and the ring, making it hydrophobic.

The polyalkylene oxides may be grafted with a vinyl ester as the monomer of component (c). The vinyl ester may be derived from a saturated monocarboxylic acid, which may contain 1 to 6 carbon atoms, or from 1 to 3 carbon atoms, or from 1 to 2 carbon atoms, or 1 carbon atom. Suitable vinyl esters may be selected from the group consisting of vinyl formate, vinyl acetate, vinyl propionate, vinyl butyrate, vinyl valerate, vinyl iso-valerate, vinyl caproate, or mixtures thereof. Preferred monomers of component (c) include those selected from the group consisting of vinyl acetate, vinyl propionate, or mixtures thereof, preferably vinyl acetate.

Conventionally, molecular weights are expressed by their “K-values,” which are derived from relative viscosity measurements. The graft copolymers may have a K value of from 5.0 to 200, optionally from 5.0 to 50, determined according to H. Fikentscher in 2% strength by weight solution in dimethylformamide at 25 C.

Particularly preferred graft copolymers of the present invention have a polyethylene oxide backbone grafted with one or more side chains of polyvinyl acetate. More preferably, the weight ratio of the polyethylene oxide backbone over the polyvinyl acetate side chains ranges from about 1:0.2 to about 1:10, or from about 1:0.5 to about 1:6, and most preferably from about 1:1 to about 1:5. One example of such preferred amphiphilic graft copolymers is the Sokalan™ HP22 polymer, which is commercially available from BASF Corporation. This polymer has a polyethylene oxide backbone grafted with polyvinyl acetate side chains. The polyethylene oxide backbone of this polymer has a number average molecular weight (Mn) of about 6,000 g/mol (equivalent to about 136 ethylene oxide units), and the weight ratio of the polyethylene oxide backbone over the polyvinyl acetate side chains is about 1:3. The number average molecular weight (Mn) of this polymer itself is about 24,000 g/mol.

### 3. SOLID PARTICLES

The solid particles contained in the loading composition according to the present invention may comprise an oxidative dye compound, a pH modifier and/or a buffering agent, a radical scavenger, a chelant, a warming active, a color indicator, an anionic surfactant, an enzyme, a bleaching agent, an effervescent system or any combinations thereof. In some embodiments, the solid particles have a preferred average particle size of from 80 μm to 2000 μm. Particularly, the particles may have an average particle size of from 90 μm to 1000 μm, preferably from 100 μm to 700 μm, more preferably from 110 μm to 500 μm, and most preferably from 120 μm to 400 μm, for example 100 μm, 200 μm, 300 μm, 400 μm, 500 μm, 600 μm, 700 μm, 800 μm, 900 μm, 1000 μm, 1100 μm, 1200 μm or any ranges therebetween. Particularly, the solid particles may be characterized by a bulk density of from 250 g/l to 500 g/l, e.g., from 300 g/l to 500 g/l, from 350 g/l to 500 g/l.

The loading composition may comprise from 1% to 99%, preferably from 5% to 90%, more preferably from 30% to 70%, for example 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95% or any ranges therebetween, of the solid particles by total weight of the loading composition.

### a) Oxidative Dye Compounds

The solid particles contained in the loading composition according to the present invention may comprise an oxidative dye compound in the form of primary intermediates or couplers.

These compounds are well known in the art, and include aromatic diamines, aminophenols, aromatic diols and their derivatives (a representative but not exhaustive list of oxidation dye precursor can be found in Sagarin, “Cosmetic Science and Technology”, “Interscience, Special Edn. Vol. 2 pages 308 to 310). It is to be understood that the precursors detailed below are only by way of example and are not intended to limit the compositions and processes herein. These are: 1,7-Dihydroxynaphthalenc (1,7-NAPHTHALENEDIOL), 1,3-Diaminobenzene (m-PHENYLENEDIAMINE), 1-Methyl-2,5-diaminobenzene (TOLUENE-2,5-DIAMINE), 1,4-Diaminobenzenc (p-PHENYLENEDIAMINE), 1,3-Dihydroxybenzene (RESORCINOL), 1,3-Dihydroxy-4-chlorobenzene, (4-CHLORORESORCINOL), 1-Hydroxy-2-aminobenzene, (o-AMINOPHENOL), 1-Hydroxy-3-aminobenzene (m-AMINOPHENOL), 1-Hydroxy-4-amino-benzene (p-AMINOPHENOL), 1-Hydroxynaphthalenc (1-NAPHTHOL), 1,5-Dihydroxynaphthalene (1,5-NAPHTHALENEDIOL), 2,7-dihydroxynaphthalene (2,7-NAPHTHELENEDIOL) 1-Hydroxy-2,4-diaminobenzene (4-DIAMINOPHENOL), 1,4-Dihydroxybenzene (HYDROQUINONE), 1-Hydroxy-4-methylaminobenzene (p-METHYLAMINOPHENOL), 6-Hydroxybenzo-morpholine (HYDROXYBENZOMORPHOLINE), 1-Methyl-2-hydroxy-4-aminobenzene (4-AMINO-2-HYDROXY-TOLUENE), 3,4-Diaminobenzoic acid (3,4-DIAMINOBENZOIC ACID), 1-Methyl-2-hydroxy-4-(2′-hydroxyethyl)aminobenzene (2-METHYL-5-HYDROXY-ETHYLAMINO-PHENOL), 1,2,4-Trihydroxybenzene (1,2,4-TRIHYDROXYBENZENE), 1-Phenol-3-methylpyrazol-5-on (PHENYLMETHYLPYRAZOLONE), 1-(2′-Hydroxyethyloxy)-2,4-diaminobenzene (2,4-DIAMINOPHENOXY-ETHANOL HCL), 1-Hydroxy-3-amino-2,4-dichlorobenzene (3-AMINO-2,4-DICHLORO-PHENOL), 1,3-Dihydroxy-2-methylbenzene (2-METHYLRESORCINOL), 1-Amino-4-bis-(2′-hydroxyethyl)aminobenzene (N,N-BIS(2-HYDROXY-ETHYL)-p-PHENYLENE-DIAMINE), 2,4,5,6-Tetraaminopyrimidine (HC Red 16), 1-Hydroxy-3-methyl-4-aminobenzene (4-AMINO-m-CRESOL), 1-Hydroxy-2-amino-5-methylbenzene (6-AMINO-m-CRESOL), 1,3-Bis-(2,4-Diaminophenoxy)propane (1,3-BIS-(2,4-DIAMINO-PHENOXY)-PROPANE), 1-(2′-Hydroxyethyl)-2,5-diaminobenzene (HYDROXYETHYL-p-PHENYLENE DIAMINE SULPHATE), 1-Methoxy-2-amino-4-(2′-hydroxyethylamino)benzene, (2-AMINO-4-HYDROXYETHYLAMINOANISOLE) 1-Hydroxy-2-methyl-5-amino-6-chlorobenzene (5-AMINO-6-CHLORO-o-CRESOL), 1-Hydroxy-2-amino-6-methylbenzene (6-AMINO-o-CRESOL), 1-(2′-Hydroxyethyl)-amino-3,4-methylenedioxybenzene (HYDROXYETHYL-3,4-METHYLENEDIOXY-ANILINE HCl), 2,6-Dihydroxy-3,4-dimethylpyridine (2,6-DIHYDROXY-3,4-DIMETHYLPYRIDINE), 3,5-Diamino-2,6-dimethoxypyridine (2,6-DIMETHOXY-3,5-PYRIDINEDIAMINE), 5,6-Dihydroxyindole (,DIHYDROXY-INDOLE), 4-Amino-2-aminomethylphenol (2-AMINOETHYL-p-AMINO-PHENOL HCl), 2,4-Diamino-5-methylphenetol (2,4-DIAMINO-5-METHYL-PHENETOLE HCl), 2,4-Diamino-5-(2′-hydroxyethyloxy)toluene (2,4-DIAMINO-5-METHYLPHENOXYETHANOL HCl), 5-Amino-4-chloro-2-methylphenol (5-AMINO-4-CHLORO-o-CRESOL), 4-Amino-1-hydroxy-2-(2′-hydroxyethylaminomethyl)benzene HYDROXYETHYLAMINOMETHYL-p-AMINO PHENOL HCl), 4-Amino-1-hydroxy-2-methoxymethylbenzene (2-METHOXYMETHYL-p-AMINOPHENOL HCl), 1,3-Bis(N(2-Hydroxyethyl)N(4-amino-phenyl)amino-2-propanol (HYDROXYPROPYL-BIS-(N-HYDROXY-ETHYL-p-PHENYLENEDIAMINE)HCL), 6-Hydorxyindole (6-HYDROXY-INDOLE), 2,3-Indolinedione (ISATIN), 3-Amino-2-methylamino-6-methoxypyridine (HC BLUE NO. 7), 1-Phenyl-3-methyl-5-pyrazolone-2,4-dihydro-5,2-phenyl-3H-pyrazole-3-one, 2-Amino-3-hydroxypyridine (2-AMINO-3-HYDROXYPYRIDINE), 5-Amino-salicylic acid, 1-Methyl-2,6-bis(2-hydroxy-ethylamino)benzene (2,6-HYDROXYETHYLAMINO-TOLUENE), 4-Hydroxy-2,5,6-triaminopyrimidine (2,5,6-TRIAMINO-4-PYRIMIDINOL SULPHATE), 2,2′-[1,2-Ethanediyl-bis-(oxy-2,1-ethanediyloxy)]-bis-benzene-1,4-diamine (PEG-3,2′,2′-DI-p-PHENYLENEDIAMINE), 5,6-Dihydroxyindoline (DIHYDROXYINDOLINE), N,N-Dimethyl-3-urcidoaniline (m-DIMETHYL-AMINO-PHENYLUREA), 2,4-Diamino-5-fluortoluenesulfatehydrate (4-FLUORO-6-METHYL-m-PHENYLENEDIAMINE SULPHATE) and 1-Acetoxy-2-methylnaphthalene (1-HYDROXYYETHYL-4,5-DIAMINOPYRAZOLE SULPHATE). These can be used in the molecular form or in the form of peroxide-compatible salts.

### b) pH Modifiers and Buffering Agents

The solid particles contained in the loading composition according to the present invention may comprise a pH modifier and/or a buffering agent in an amount that is sufficiently effective to adjust the pH of the composition to fall within a range from about 3 to about 13, in some embodiments from about 8 to about 12, and even from about 8 to about 11. Suitable pH modifiers and/or buffering agents for use herein include, but are not limited to: ammonia, alkanolamines such as monoethanolamine, diethanolamine, triethanolamine, monopropanolamine, dipropanolamine, tripropanolamine, tripropanolamine, 2-amino-2-methyl-1-propanol, and 2-amino-2-hydroxymethyl-1,3,-propandiol and guanidium salts, alkali metal and ammonium hydroxides and carbonates, preferably sodium hydroxide and ammonium carbonate, and acidulents such as inorganic and inorganic acids, e.g., phosphoric acid, acetic acid, ascorbic acid, citric acid or tartaric acid, hydrochloric acid, and mixtures thereof.

### c) Radical Scavenger System

The solid particles contained in the loading composition according to the present invention may comprise a radical scavenger in a sufficient amount to reduce damage to the hair during an oxidative bleaching or coloring process. The radical scavenger is preferably selected such that it is not an identical species as the alkalizing agent. Preferred radical scavengers may be selected from the classes of alkanolamines, amino sugars, amino acids and mixtures thereof, and may include, but are not limited to: monoethanolamine, 3-amino-1-propanol, 4-amino-1-butanol,5-amino-1-pentanol, 1-amino-2-propanol, 1-amino-2-butanol, 1-amino-2-pentanol, 1-amino-3-pentanol, 1-amino-4-pentanol, 3-amino-2-methylpropan-1-ol, 1-amino-2-methylpropan-2-ol, 3-aminopropane-1,2-diol, glucosamine, N-acetylglucosamine, glycine, arginine, lysine, proline, glutamine, histidine, serine, tryptophan and potassium, sodium and ammonium salts of the above and mixtures thereof. Other preferred radical scavenger compounds include benzylamine, glutamic acid, imidazole, di-tert-butylhydroxytoluene, hydroquinone, catechol and mixtures thereof.

### d) Chelants

The solid particles contained in the loading composition according to the present invention may comprise a chelant in an amount sufficient to reduce the amount of metals available to interact with formulation components. Suitable chelants for use herein include but are not limited to: diamine-N,N′-dipolyacid, monoamine monoamide-N,N′-dipolyacid, and N,N′-bis(2-hydroxybenzyl)ethylenediamine-N,N′-diacetic acid chelants (preferably EDDS (ethylenediaminedisuccinic acid)), carboxylic acids (preferably aminocarboxylic acids), phosphonic acids (preferably aminophosphonic acids) and polyphosphoric acids (in particular straight polyphosphoric acids), their salts and derivatives.

### c) Warming Actives

The solid particles contained in the loading composition according to the present invention may comprise a warming active. The warming actives may include heat generating agents, or heat generating powders which release heat via exothermic reactions (heat producing) when they are mixed with water during application. The heat generating agents include, but are not limited to, inorganic salts, glycols, finely divided solid adsorbent materials, and iron redox systems. In one embodiment the warming actives are selected from the group consisting of anhydrous inorganic salts including, but not limited to calcium chloride, magnesium chloride, calcium oxide, magnesium sulphate, aluminium sulphate and combinations thereof. In yet another embodiment the warming actives of the present invention are selected from the group consisting of anhydrous calcium chloride, anhydrous magnesium chloride, anhydrous magnesium sulphate, and combinations thereof.

### f) Color Indicators

The solid particles contained in the loading composition according to the present invention may comprise a color indicator. Such color indicators can be present in an amount sufficient to result in a visual color change when the indicator is contacted with water. The term “visual color change” refers to a color change that can be detected by the human eye, either alone, or with the aid of an energy source such as a black light. The color indicators of the present invention can include, but are not limited to, those selected from the group consisting of pH indicators, photoactive pigments, thermochromatic pigments, and combinations thereof.

In one embodiment the color change is a pH sensitive color changing component. The color indicators can be selected from the group consisting of bromocresol green, phenolphthalein, σ-cresolphthalein, thymolphthalein, coumarin, 2,3-dioxyxanthone, coumeric acid, 6,8-dinitro-2,4(1H) quinazolinedione, ethyl-bis (2,4-dimethylphenyl) ethanoate, and combinations thereof.

### g) Enzyme

The solid particles contained in the loading composition according to the present invention may comprise an enzyme. Any enzyme known in the art can be used in the loading composition. A preferred enzyme is selected from the group consisting of proteases, amylases, cellulases, lipases, xylogucanases, pectate lyases, mannanases, cutinases, and any combinations thereof.

### h) Bleaching Agent

The solid particles contained in the loading composition according to the present invention may comprise a bleaching agent. The bleaching agent may be selected from the group consisting of a source of available oxygen, a bleach activator, a pre-formed peracid, a bleach catalyst, a reducing bleach, and any combinations thereof. Particularly, the bleaching agent may be in a form of particles that preferably have an average particle size of from 80 μm to 2000 μm, preferably from 100 μm to 1500 μm, for example 100 μm, 200 μm, 300 μm, 400 μm, 500 μm, 600 μm, 700 μm, 800 μm, 900 μm, 1000 μm, 1100 μm, 1200 μm or any ranges therebetween. A preferred range of particle size may bring about an improved dissolution profile and/or an improved leakage performance.

The source of available oxygen (AvOx) may be a source of hydrogen peroxide that is preferably selected from the group consisting of percarbonate salts, perborate salts, persulfate salts and any combinations thereof. The source of available oxygen may be at least partially coated, or even completely coated, by a coating ingredient such as a carbonate salt, a sulphate salt, a silicate salt, borosilicate, or any mixture thereof, including mixed salts thereof. Suitable percarbonate salts can be prepared by a fluid bed process or by a crystallization process. Suitable perborate salts include sodium perborate mono-hydrate (PB1), sodium perborate tetra-hydrate (PB4), and anhydrous sodium perborate which is also known as fizzing sodium perborate. Other suitable sources of AvOx include persulphate, such as oxone. Another suitable source of AvOx is hydrogen peroxide

The bleach activator may be selected from the group consisting of tetraacetylethylenediamine (TAED); oxybenzene sulphonates such as nonanoyl oxybenzene sulphonate (NOBS), caprylamidononanoyl oxybenzene sulphonate (NACA-OBS), 3,5,5-trimethyl hexanoyloxybenzene sulphonate (Iso-NOBS), dodecyl oxybenzene sulphonate (LOBS), and any mixture thereof; caprolactams; pentaacetate glucose (PAG); nitrile quaternary ammonium; imide bleach activators, such as N-nonanoyl-N-methyl acetamide; and any mixture thereof.

The pre-formed peracid may be N,N-pthaloylamino peroxycaproic acid (PAP).

The bleach catalyst may be selected from the group consisting of oxaziridinium-based bleach catalysts, transition metal bleach catalysts, and any combinations thereof.

A suitable oxaziridinium-based bleach catalyst has the formula:

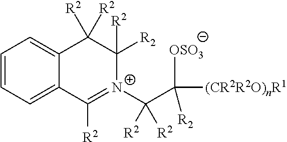

wherein: R¹ is selected from the group consisting of: H, a branched alkyl group containing from 3 to 24 carbons, and a linear alkyl group containing from 1 to 24 carbons; R¹ can be a branched alkyl group comprising from 6 to 18 carbons, or a linear alkyl group comprising from 5 to 18 carbons, R¹ can be selected from the group consisting of: 2-propylheptyl, 2-butyloctyl, 2-pentylnonyl, 2-hexyldecyl, n-hexyl, n-octyl, n-decyl, n-dodecyl, n-tetradecyl, n-hexadecyl, n-octadecyl, iso-nonyl, iso-decyl, iso-tridecyl and iso-pentadecyl; R² is independently selected from the group consisting of: H, a branched alkyl group comprising from 3 to 12 carbons, and a linear alkyl group comprising from 1 to 12 carbons; optionally R² is independently selected from H and methyl groups; and n is an integer from 0 to 1.

Transition metal bleach catalyst may comprise copper, iron, titanium, ruthenium, tungsten, molybdenum, and/or manganese cations. Suitable transition metal bleach catalysts are manganese-based transition metal bleach catalysts.

The reducing bleach may be sodium sulphite and/or thiourea dioxide (TDO).

Particularly, the loading composition may comprise from 1% to 99%, preferably from 10% to 80%, more preferably from 30% to 70%, for example 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95% or any ranges therebetween, of the bleaching agent by total weight of the loading composition. More particularly, the loading composition may comprise from 1% to 99%, preferably from 10% to 80%, more preferably from 30% to 70%, for example 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95% or any ranges therebetween, of the source of available oxygen by total weight of the loading composition, and/or the loading composition may comprise from 1% to 99%, preferably from 10% to 80%, more preferably from 30% to 70%, for example 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95% or any ranges therebetween, of the bleach activator by total weight of the loading composition.

### i) Effervescent System

The solid particles contained in the loading composition according to the present invention may comprise an effervescent system. Any effervescent system known in the art can be used in the loading composition. A preferred effervescent system comprises an acid source and an alkali source, capable of reacting with each other in the presence of water to produce a gas.

The acid source component may be any organic, mineral or inorganic acid, or a derivative thereof, or a combination thereof. Preferably the acid source component comprises an organic acid. The acid compound is preferably substantially anhydrous or non-hygroscopic and the acid is preferably water-soluble. It may be preferred that the acid source is overdried.

Suitable acids source components include citric acid, malic acid, tartaric acid, fumaric acid, adipic acid, maleic acid, aspartic acid, glutaric acid, malonic acid, succinic acid, boric acid, benzoic acid, oleic acid, citramalic acid, 3-chetoglutaric acid or any combinations thereof. Citric acid, maleic or tartaric acid are especially preferred. The acid source may be further coated with a coating such as a salt. In an embodiment, citric acid as the acid source may be coated with sodium citrate.

Any alkali source which has the capacity to react with the acid source to produce a gas may be present in the particle, which may be any gas known in the art, including nitrogen, oxygen and carbon dioxide gas. Preferred can be an alkali source that is selected from the group consisting of a carbonate salt, a bicarbonate salt, a sesquicarbonate salt and any combinations thereof. The alkali source is preferably substantially anhydrous or non-hydroscopic. It may be preferred that the alkali source is overdried.

Preferably this gas is carbon dioxide, and therefore the alkali source is a preferably a source of carbonate, which can be any source of carbonate known in the art. In a preferred embodiment, the carbonate source is a carbonate salt. Examples of preferred carbonates are the alkaline earth and alkali metal carbonates, including sodium or potassium carbonate, bicarbonate and sesqui-carbonate and any combinations thereof with ultra-fine calcium carbonate or sodium carbonate. Alkali metal percarbonate salts are also suitable sources of carbonate species, which may be present combined with one or more other carbonate sources.

### 4. ADDITIONAL INGREDIENTS

In addition to the above-described ingredients, the loading composition of the present invention may comprise one or more additional ingredients, depending on its intended application. Such one or more additional ingredients may be selected from the group consisting of fabric care actives, dishwashing actives, hard surface cleaning actives, beauty and/or skin care actives, personal cleansing actives, hair care actives, oral care actives, feminine care actives, baby care actives, a bittering agent and any combinations thereof.

Particularly, the loading composition may further comprise an additional ingredient selected from the group consisting of a softening agent, silicone, an emulsifier, an enzyme, a colorant, a brightener, a dye transfer inhibiting agent, a deposition aid, an anti-microbial agent, a chelant, a non-film forming polymer, an anti-foamer, a defoamer, and any combinations thereof.

The loading composition may comprise from 0.0001% to 99%, preferably from 1% to 95%, more preferably from 10% to 80%, for example 0.001%, 0.01%, 0.1%, 1%, 2%, 3%, 4%, 5%, 8%, 10%, 15%, 20%, 25%, 30%, 35%, 40%, 45%, 50%, 55%, 60%, 65%, 70%, 75%, 80%, 85%, 90%, 95% or any ranges therebetween, of the additional ingredient by total weight of the loading composition.

### V. CONVERSION OF WATER-SOLUBLE SHEET AND LOADING COMPOSITION INTO DISSOLVABLE UNIT DOSE ARTICLES CONTAINING LOADING COMPOSITION

Once the water-soluble sheet of the present invention is formed, as described hereinabove, such sheet can be treated by loading the loading composition to form dissolvable unit dose articles of any desirable three-dimensional shapes, including but not limited to: spherical, cubic, rectangular, oblong, cylindrical, rod, sheet, flower-shaped, fan-shaped, star-shaped, disc-shaped, and the like. The sheets can be treated by any means known in the art, examples of which include but are not limited to, chemical means, mechanical means, and combinations thereof. Such treatment steps are hereby collectively referred to as a “conversion” process, i.e., which functions to convert such water-soluble sheet of the present invention into a dissolvable unit dose article containing a loading composition.

In an embodiment of the present disclosure, the loading composition can be added onto one surface of one water-soluble sheet and then the water-soluble sheet is folded towards the surface loaded the loading composition so that the loading composition is contained between the two halves of the folded sheet. Preferably, the folded sheet can be further treated in an edge sealing process to prevent the leakage of the loading composition.

In another embodiment of the present disclosure, the dissolvable unit dose article may be a multilayer dissolvable unit dose article in which the loading composition can be added between adjacent sheets. Furthermore, the multilayer dissolvable unit dose articles of the present invention may be characterized by a maximum dimension D and a minimum dimension z (which is perpendicular to the maximum dimension), while the ratio of D/z (hereinafter also referred to as the “Aspect Ratio”) ranges from 1 to about 10, preferably from about 1.4 to about 9, preferably from about 1.5 to about 8, more preferably from about 2 to about 7. Note that when the Aspect Ratio is 1, the dissolvable solid article has a spherical shape. When the Aspect Ratio is about 1.4, the dissolvable solid article has a cubical shape. The multilayer dissolvable solid article of the present invention may have a minimal dimension z that is greater than about 3 mm but less than about 20 cm, preferably from about 4 mm to about 10 cm, more preferably from about 5 mm to about 30 mm.

The above-described multilayer dissolvable unit dose article may comprise more than two of sheets. For example, it may comprise from about 3 to about 50, preferably from about 4 to about 40, more preferably from about 5 to about 30, for example 6, 7, 8, 9, 10, 15, 20, 25, 30 or any ranges therebetween, of the sheets.

In some preferred embodiments, the multiple layers of the multilayer dissolvable unit dose article may be bonded together through heat-compressing when a loading composition is contained between layers. In some other embodiments, the multilayer dissolvable unit dose article may further comprise water-soluble thread at the edges which may help to seal the multiple layers together.

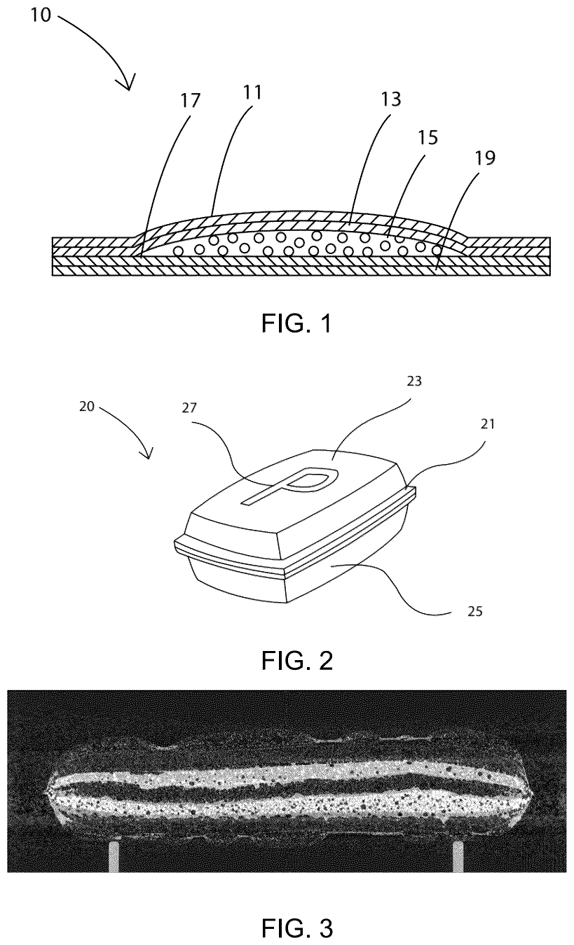

FIG. 1 shows an exemplary dissolvable unit dose article 10, which comprises flexible, dissolvable, porous sheets 11, 13, 17 and 19 as well as a paste 15 loaded between sheets 13 and 17. The paste 15 comprises a non-aqueous liquid carrier, solid particles and a polyalkylene polymer. The dissolvable unit dose article 10 can be made with or without an edge sealing step.

FIG. 2 shows another exemplary dissolvable unit dose article 20, which comprises flexible, dissolvable, porous sheets 23 and 25. The dissolvable unit dose article 20 comprises an edge-seal 21 and an embossed pattern 27.

In a preferred embodiment, the dissolvable unit dose article comprises 2, 3, 4, 5, 6, 7, 8, 9, 10 or more water-soluble sheets in which one or more pastes are loaded between adjacent sheets. For example, the dissolvable unit dose article comprises 5 sheets in which two pastes are respectively loaded between 2^{nd} and 3^{rd} sheets and between 3^{rd} and 4^{th} sheets. In another embodiment, the dissolvable unit dose article may further comprise a loading composition in a form of powders. Preferably, the loading composition in a form of powders is characterized by a bulk density of from 250 g/l to 500 g/l, e.g., from 300 g/l to 500 g/l, from 350 g/l to 500 g/l. For example, FIG. 3 shows another exemplary dissolvable unit dose article comprising 5 sheets in which a first loading composition in a form of paste is loaded between 2^{nd} and 3^{rd} sheets and a second loading composition in a form of powders is loaded between 3^{rd} and 4^{th} sheets. Without wishing to be bound by theory, it is believed that loading composition in a form of powders having a relatively low bulk density would result in an improved leakage performance compared to powders having a relatively high bulk density.

Particularly, the loading composition may be applied between individual sheets of the multilayer dissolvable solid article by any appropriate means, e.g., by spraying, sprinkling, dusting, coating, spreading, dipping, injecting, rolling, or even vapor deposition. More particularly, the loading composition may be applied on one or both of contacting surfaces of adjacent sheets in the stack. In a preferred embodiment, in order to avoid interference of the loading composition with the cutting seal or edge seal near the peripherals of the individual sheets, the loading composition may be applied in a central region on each of the applied surfaces of the respective sheets, which is preferably defined as a region that is spaced apart from the peripherals of such adjacent sheets by a distance that is at least 5%, preferably at least 10%, more preferably at least 15%, most preferably at least 20%, of the maximum Dimension D. In an alternative preferred embodiment, said loading composition is applied throughout the applied surfaces of the respective sheets, preferably wherein the applied area accounts for at least 90%, preferably 95%, more preferably 98%, most preferably 99% of the total area of the applied surfaces.

In a preferred embodiment, the loading composition may be applied on one or both contacting surfaces of any adjacent sheets in the solid article. In another preferred embodiment, the loading composition may be applied on one or both contacting surfaces of middle two sheets in the stack. In yet another preferred embodiment, the loading composition may be applied on one or both of contacting surfaces of any two adjacent sheets in the stack excluding the two outermost sheets.

In some preferred embodiments, the sheet in the unit dose article according to the present disclosure is relatively high compressible. Without wishing to be bound by theory, it is believed that the sheet being relatively high compressible would result in an improved leakage performance compared to the sheet being relatively low compressible.

The conversion process according to the present disclosure may be achieved by a system for preparing dissolvable unit dose articles according to the present disclosure. A typical system for preparing dissolvable unit dose articles according to the present disclosure may comprise a belt conveyor, a loading unit, an edge scaling unit, and optionally, an embossing unit. Particularly, the loading unit may comprise a nozzle and a flattening mechanism in which the nozzle is configured to load a loading composition in a form of paste onto a sheet and the flattening mechanism is configured to spread the loading composition evenly on the sheet. The loading unit may comprise a dispenser and a spreading roller in which the dispenser is configured to load a loading composition in a form of powders onto a sheet and the spreading roller is configured to spread the loading composition evenly on the sheet.

In some embodiments, the system may further comprise one or more compression rollers which are configured to compress the stack of the sheets. Particularly, the compression rollers can be located at anywhere after two sheets are fed on the belt conveyor.

In some embodiments, the system may further comprise one or more spraying units which are configured to spray an aqueous liquid (e.g. water) onto sheets. The addition of the aqueous liquid may be helpful in combining multiple sheets into a dissolvable unit dose article and improve the leakage of loading compositions.

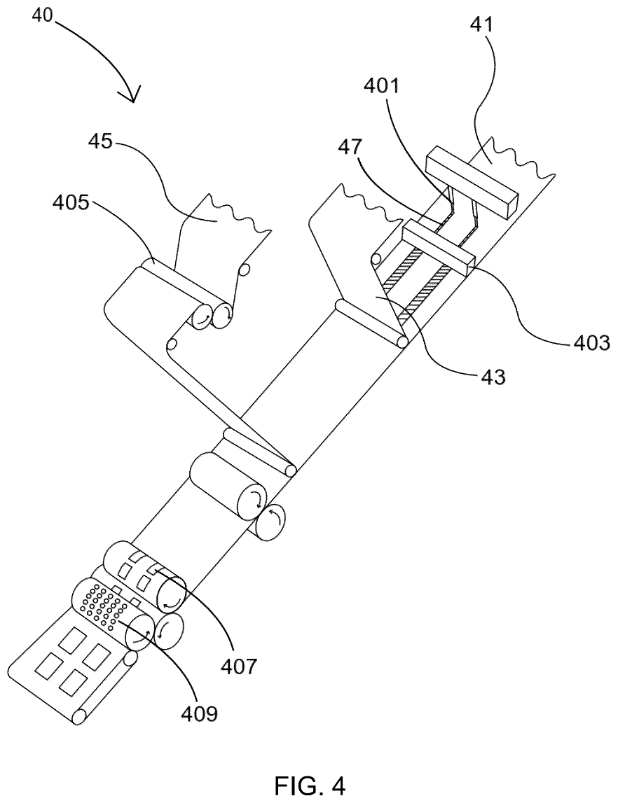

FIG. 4 shows an exemplary system 40 for preparing dissolvable unit dose articles comprising a belt conveyor, a loading unit, an edge sealing unit which is configured to seal edges of the unit dose articles, and an embossing unit which is configured to emboss a pattern on the surface of sheets. Particularly, a first flexible, dissolvable, porous sheet 41, and a second flexible, dissolvable, porous sheet 43, as well as, optionally, a third flexible, dissolvable, porous sheet 45 are respectively fed on the belt conveyor sequentially. In some embodiments, the third sheet is fed so that the unit dose articles obtained has three layers in total. In some other embodiments, the third sheet is not fed so that the unit dose articles obtained has two layers in total. The loading unit comprises a nozzle 401 through which a loading composition 47 in a form of paste may be loaded onto the sheet 41, and a flattening mechanism 403 by which the loading composition 47 is spread evenly on the sheet 41. Then, the second flexible, dissolvable, porous sheet 43 is fed on top of the first flexible, dissolvable, porous sheet 41 so that the loading composition 47 is contained between the sheets 41 and 43. Subsequently, if present, the third flexible, dissolvable, porous sheet 45 passes through the embossing unit comprising an embossing roller 405 and then fed on top of the sheet 43 to provide a stack of sheets. If the third sheet is not present, a stack of sheets comprising the first and the second sheets as well as the loading composition therebetween is provided. Finally, the stack of sheets pass through the edge scaling unit comprising an edge scaling roller 407 and a transferring roller 409. The edge sealing roller 407 comprises a tooling having a specific shape which can apply heat and pressure onto the stack of sheets so that the stack of sheets can be cut into unit dose articles with sealed edge. The transferring roller 409 is configured to transfer the unit dose articles to a next station through vacuum applied onto the transferring roller 409.

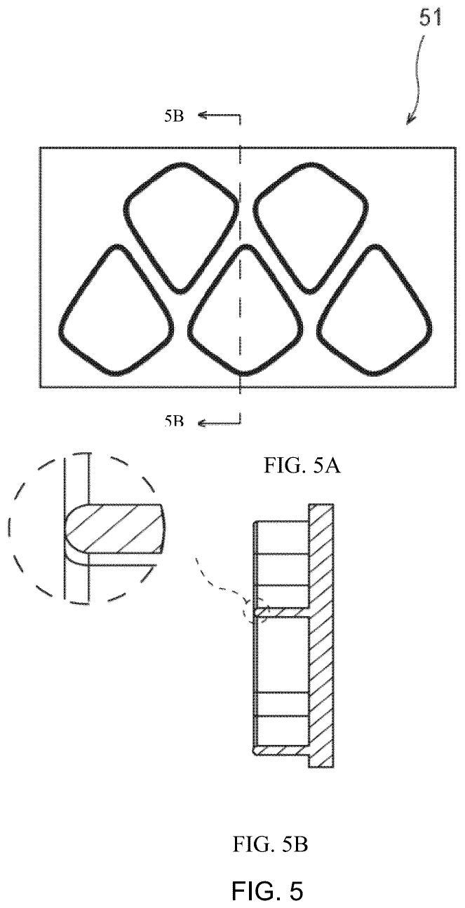

FIG. 5 shows an exemplary tooling 51 used to form the edge seal of the article which can be made of stainless steel. FIG. 5A shows a top view of tooling 51 used to produce five unit dose articles. FIG. 5B shows a cross-sectional view of the tooling bounded by the letters 5B-5B in FIG. 5A as well as a cross-sectional detail of a tip of the tooling that comes in contact with the sheets to be sealed. Preferably, this tooling 51 may be bolted to a heated mechanical press which can apply both heating and pressure onto the sheets to be sealed. The heated mechanical press can be configured to apply a temperature of 50° C., 60° C., 70° C., 80° C., 90° C., 100° C., 110° C., 120° C., 130° C., 140° C., 150° C., 160° C., 170° C., 180° C., 190° C. or any ranges therebetween, and a pressure of 100 psi, 500 psi, 1000 psi, 3000 psi, 5000 psi, 7000 psi, 9000 psi, 10000 psi, 15000 psi, 20000 psi or any ranges therebetween. The duration of the contacting between the tooling 51 and the sheets can be set as 0.05, 0.1, 0.2, 0.3, 0.4, 0.5, 0.8, 1, 2, 3, 4, 5, 6, 7, 8, 9, 10 seconds or any ranges therebetween. The edge sealing can be one-step or two-step approach. In the two-step approach, the heated mechanical press can apply a first temperature and a first pressure in a first step to allow the edge scaling and then a second temperature and a second pressure in a second step to cut the unit dose articles out of the rest of the sheets. In some embodiments, the edge seal breadth can be half of the width of the tooling tip. For example, the width of the tip is 0.3 cm, and the edge seal breadth would be 0.15 cm. In some particular embodiments, the edge seal breadth can be 0.05 cm, 0.1 cm, 0.15 cm, 0.2 cm, 0.25 cm, 0.3 cm, 0.35 cm, 0.4 cm, 0.45 cm, 0.5 cm and any ranges therebetween.

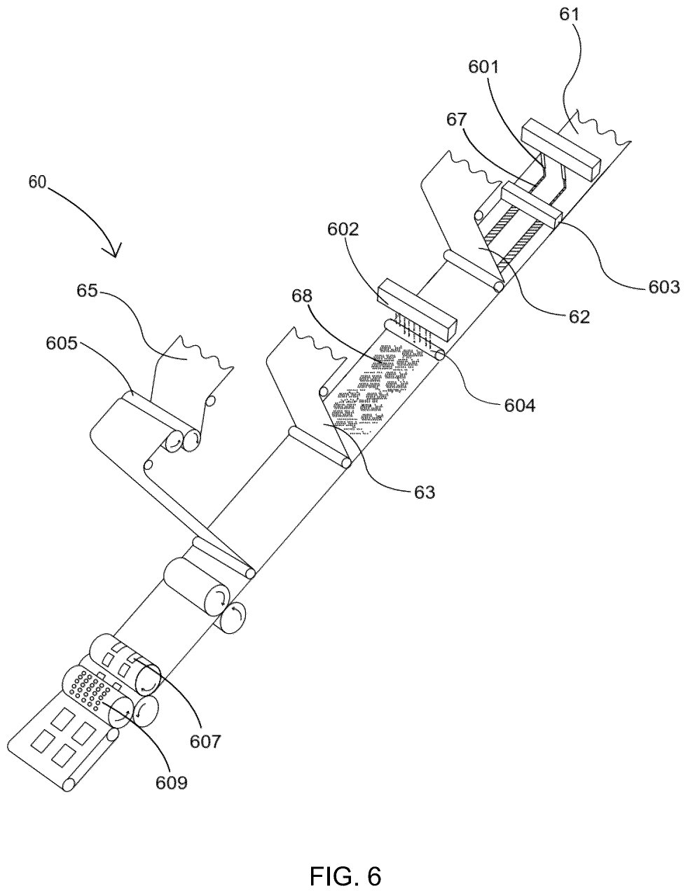

FIG. 6 shows another exemplary system 60 for preparing dissolvable unit dose articles comprising a belt conveyor, a loading unit, an edge sealing unit which is configured to seal edges of the unit dose articles, and an embossing unit which is configured to emboss a pattern on the surface of sheets. Particularly, a first flexible, dissolvable, porous sheet 61, a second flexible, dissolvable, porous sheet 62, a third flexible, dissolvable, porous sheet 63 and a fourth flexible, dissolvable, porous sheet 65 are respectively fed on the belt conveyor sequentially. The loading unit comprises a nozzle 601 through which a first loading composition 67 in a form of paste may be loaded onto the sheet 61, and a flattening mechanism 603 by which the first loading composition 67 is spread evenly on the sheet 61. Then, the second sheet 62 is fed on top of the first sheet 61 so that the first loading composition 67 is contained between the sheets 61 and 62. The loading unit further comprises a dispenser 602 through which a second loading composition 68 in a form of powders may be loaded onto the second sheet 62 as well as a spreading roller 604 by which the second loading composition 68 is spread evenly on the second sheet 62. Then, the third sheet 63 is fed on top of the second sheet 62 so that the second loading composition 68 is contained between the sheets 62 and 63. Subsequently, the fourth sheet 65 passes through the embossing unit comprising an embossing roller 605 and then fed on top of the sheet 63. Finally, the stack of sheets 61, 62, 63 and 65 pass through the edge sealing unit comprising an edge scaling roller 607 and a transferring roller 609. The edge sealing roller 607 comprises a tooling having a specific shape which can apply heat and pressure onto the stack of sheets 61, 62, 63 and 65 so that the stack of sheets 61, 62, 63 and 65 can be cut into unit dose articles with sealed edge. The transferring roller 609 is configured to transfer the unit dose articles to a next station through vacuum applied onto the transferring roller 609.

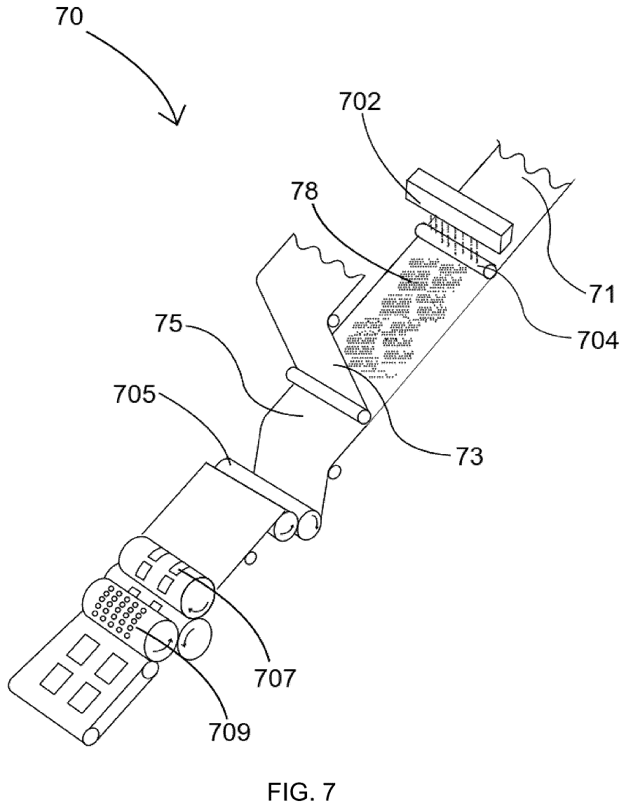

FIG. 7 shows another exemplary system for preparing dissolvable unit dose articles, wherein the system comprises a belt conveyor, a loading unit, an embossing unit which is configured to apply heat and pressure on the surface of sheets to achieve both bonding and embossing, and a cutting unit. Particularly, a first flexible, dissolvable, porous sheet 71, and a second flexible, dissolvable, porous sheet 73 are respectively fed on the belt conveyor sequentially. The loading unit comprises a dispenser 702 through which a loading composition in a form of powders may be loaded between the two sheets as well as a spreading roller 704 by which a loading composition 78 in the form of powder is spread evenly on the second sheet 73. Then, a stack 75 of sheets containing the loading composition therebetween pass through the embossing unit comprising an embossing roller 705 which can apply heat and pressure onto the stack 75 of sheets to make a pattern on the surface of the stack 75 of sheets and to make the stack of sheets bond together. Finally, the stack 75 of sheets containing the loading composition therebetween pass through the cutting unit wherein the stack of sheets can be cut into unit dose articles. The cutting unit comprises a cutting roller 707 and a transferring roller 709 which is configured to transfer the unit dose articles to a next station through vacuum applied onto the transferring roller 709.

In some other embodiments, the system for preparing dissolvable unit dose articles may further comprise a sewing unit which is configured to sew edges of dissolvable unit dose articles with water-soluble thread, e.g. water-soluble PVA thread.

### TEST METHODS

### Test 1: Scanning Electron Microscopic (SEM) Method for Determining Surface Average Pore Diameter of the Sheet Article

A Hitachi TM3000 Tabletop Microscope (S/N: 123104-04) is used to acquire SEM micrographs of samples. Samples of the solid sheet articles of the present invention are approximately 1 cm×1 cm in area and cut from larger sheets. Images are collected at a magnification of 50×, and the unit is operated at 15 kV. A minimum of 5 micrograph images are collected from randomly chosen locations across each sample, resulting in a total analyzed area of approximately 43.0 mm² across which the average pore diameter is estimated.

The SEM micrographs are then firstly processed using the image analysis toolbox in Matlab. Where required, the images are converted to grayscale. For a given image, a histogram of the intensity values of every single pixel is generated using the ‘imhist’ Matlab function.

Typically, from such a histogram, two separate distributions are obvious, corresponding to pixels of the brighter sheet surface and pixels of the darker regions within the pores. A threshold value is chosen, corresponding to an intensity value between the peak value of these two distributions. All pixels having an intensity value lower than this threshold value are then set to an intensity value of 0, while pixels having an intensity value higher are set to 1, thus producing a binary black and white image. The binary image is then analyzed using ImageJ (https://imagej.nih.gov, version 1.52a), to examine both the pore area fraction and pore size distribution. The scale bar of each image is used to provide a pixel/mm scaling factor. For the analysis, the automatic thresholding and the analyze particles functions are used to isolate each pore. Output from the analyze function includes the area fraction for the overall image and the pore area and pore perimeter for each individual pore detected.

Average Pore Diameter is defined as DA50: 50% of the total pore area is comprised of pores having equal or smaller hydraulic diameters than the DA50 average diameter.

$$Hydraulic diameter = ‘ 4 * Pore area ( {m}^{2} ) / Pore perimeter ( m ) ’ .$$

It is an equivalent diameter calculated to account for the pores not all being circular.

### Test 2: Micro-Computed Tomographic (μCT) Method for Determining Overall or Regional Average Pore Size and Average Cell Wall Thickness of the Open Cell Foams (OCF)

Porosity is the ratio between void-space to the total space occupied by the OCF. Porosity can be calculated from μCT scans by segmenting the void space via thresholding and determining the ratio of void voxels to total voxels. Similarly, solid volume fraction (SVF) is the ratio between solid-space to the total space, and SVF can be calculated as the ratio of occupied voxels to total voxels. Both Porosity and SVF are average scalar-values that do not provide structural information, such as, pore size distribution in the height-direction of the OCF, or the average cell wall thickness of OCF struts.

To characterize the 3D structure of the OCFs, samples are imaged using a μCT X-ray scanning instrument capable of acquiring a dataset at high isotropic spatial resolution. One example of suitable instrumentation is the SCANCO system model 50 μCT scanner (Scanco Medical AG, Brüttisellen, Switzerland) operated with the following settings: energy level of 45 kVp at 133 μA; 3000 projections; 15 mm field of view; 750 ms integration time; an averaging of 5; and a voxel size of 3 μm per pixel. After scanning and subsequent data reconstruction is complete, the scanner system creates a 16 bit data set, referred to as an ISQ file, where grey levels reflect changes in x-ray attenuation, which in turn relates to material density. The ISQ file is then converted to 8 bit using a scaling factor.

Scanned OCF samples are normally prepared by punching a core of approximately 14 mm in diameter. The OCF punch is laid flat on a low-attenuating foam and then mounted in a 15 mm diameter plastic cylindrical tube for scanning. Scans of the samples are acquired such that the entire volume of all the mounted cut sample is included in the dataset. From this larger dataset, a smaller sub-volume of the sample dataset is extracted from the total cross section of the scanned OCF, creating a 3D slab of data, where pores can be qualitatively assessed without edge/boundary effects.

To characterize pore-size distribution in the height-direction, and the strut-size, Local Thickness Map algorithm, or LTM, is implemented on the subvolume dataset. The LTM Method starts with a Euclidean Distance Mapping (EDM) which assigns grey level values equal to the distance each void voxel is from its nearest boundary. Based on the EDM data, the 3D void space representing pores (or the 3D solid space representing struts) is tessellated with spheres sized to match the EDM values. Voxels enclosed by the spheres are assigned the radius value of the largest sphere. In other words, each void voxel (or solid voxel for struts) is assigned the radial value of the largest sphere that that both fits within the void space boundary (or solid space boundary for struts) and includes the assigned voxel.

The 3D labelled sphere distribution output from the LTM data scan can be treated as a stack of two dimensional images in the height-direction (or Z-direction) and used to estimate the change in sphere diameter from slice to slice as a function of OCF depth. The strut thickness is treated as a 3D dataset and an average value can be assessed for the whole or parts of the subvolume. The calculations and measurements were done using AVIZO Lite (9.2.0) from Thermo Fisher Scientific and MATLAB (R2017a) from Mathworks.

### Test 3: Percent Open Cell Content of the Sheet Article

The Percent Open Cell Content is measured via gas pycnometry. Gas pycnometry is a common analytical technique that uses a gas displacement method to measure volume accurately. Inert gases, such as helium or nitrogen, are used as the displacement medium. A sample of the solid sheet article of the present invention is sealed in the instrument compartment of known volume, the appropriate inert gas is admitted, and then expanded into another precision internal volume. The pressure before and after expansion is measured and used to compute the sample article volume.

ASTM Standard Test Method D2856 provides a procedure for determining the percentage of open cells using an older model of an air comparison pycnometer. This device is no longer manufactured. However, one can determine the percentage of open cells conveniently and with precision by performing a test which uses Micromcritics' AccuPyc Pycnometer. The ASTM procedure D2856 describes 5 methods (A, B, C, D, and E) for determining the percent of open cells of foam materials. For these experiments, the samples can be analyzed using an Accupyc 1340 using nitrogen gas with the ASTM foampyc software. Method C of the ASTM procedure is to be used to calculate to percent open cells. This method simply compares the geometric volume as determined using calipers and standard volume calculations to the open cell volume as measured by the Accupyc, according to the following equation:

$$Open cell percentage = Open cell volume of sample / Geometric volume of sample * 100$$

It is recommended that these measurements be conducted by Micromeretics Analytical Services, Inc. (One Micromeritics Dr, Suite 200, Norcross, GA 30093). More information on this technique is available on. the Micromeretics Analytical Services web sites (www.particletesting.com or www.micromeritics.com), or published in “Analytical Methods in Fine particle Technology” by Clyde Orr and Paul Webb.

### Test 4: Final Moisture Content of the Sheet Article

Final moisture content of the solid sheet article of the present invention is obtained by using a Mettler Toledo HX204 Moisture Analyzer (S/N B706673091). A minimum of 1 g of the dried sheet article is placed on the measuring tray. The standard program is then executed, with additional program settings of 10 minutes analysis time and a temperature of 110° C.

### Test 5: Thickness of the Sheet Article

Thickness of the flexible, porous, dissolvable solid sheet article of the present invention is obtained by using a micrometer or thickness gage, such as the Mitutoyo Corporation Digital Disk Stand Micrometer Model Number IDS-1012E (Mitutoyo Corporation, 965 Corporate Blvd, Aurora, IL, USA 60504). The micrometer has a 1-inch diameter platen weighing about 32 grams, which measures thickness at an application pressure of about 0.09 psi (6.32 gm/cm²).

The thickness of the flexible, porous, dissolvable solid sheet article is measured by raising the platen, placing a section of the sheet article on the stand beneath the platen, carefully lowering the platen to contact the sheet article, releasing the platen, and measuring the thickness of the sheet article in millimeters on the digital readout. The sheet article should be fully extended to all edges of the platen to make sure thickness is measured at the lowest possible surface pressure, except for the case of more rigid substrates which are not flat.

### Test 6: Basis Weight of the Sheet Article

Basis Weight of the flexible, porous, dissolvable solid sheet article of the present invention is calculated as the weight of the sheet article per area thereof (grams/m²). The area is calculated as the projected area onto a flat surface perpendicular to the outer edges of the sheet article. The solid sheet articles of the present invention are cut into sample squares of 10 cm×10 cm, so the area is known. Each of such sample squares is then weighed, and the resulting weight is then divided by the known area of 100 cm² to determine the corresponding basis weight.

For an article of an irregular shape, if it is a flat object, the area is thus computed based on the area enclosed within the outer perimeter of such object. For a spherical object, the area is thus computed based on the average diameter as 3.14×(diameter/2)². For a cylindrical object, the area is thus computed based on the average diameter and average length as diameter x length. For an irregularly shaped three-dimensional object, the area is computed based on the side with the largest outer dimensions projected onto a flat surface oriented perpendicularly to this side. This can be accomplished by carefully tracing the outer dimensions of the object onto a piece of graph paper with a pencil and then computing the area by approximate counting of the squares and multiplying by the known area of the squares or by taking a picture of the traced area (shaded-in for contrast) including a scale and using image analysis techniques.

### Test 7: Density of the Sheet Article

Density of the flexible, porous, dissolvable solid sheet article of the present invention is determined by the equation: Calculated Density=Basis Weight of porous solid/(Porous Solid Thickness×1,000). The Basis Weight and Thickness of the dissolvable porous solid are determined in accordance with the methodologies described hereinabove.

### Test 8: Specific Surface Area of the Sheet Article

The Specific Surface Area of the flexible, porous, dissolvable solid sheet article is measured via a gas adsorption technique. Surface Area is a measure of the exposed surface of a solid sample on the molecular scale. The BET (Brunauer, Emmet, and Teller) theory is the most popular model used to determine the surface area and is based upon gas adsorption isotherms. Gas Adsorption uses physical adsorption and capillary condensation to measure a gas adsorption isotherm. The technique is summarized by the following steps; a sample is placed in a sample tube and is heated under vacuum or flowing gas to remove contamination on the surface of the sample. The sample weight is obtained by subtracting the empty sample tube weight from the combined weight of the degassed sample and the sample tube. The sample tube is then placed on the analysis port and the analysis is started. The first step in the analysis process is to evacuate the sample tube, followed by a measurement of the free space volume in the sample tube using helium gas at liquid nitrogen temperatures. The sample is then evacuated a second time to remove the helium gas. The instrument then begins collecting the adsorption isotherm by dosing krypton gas at user specified intervals until the requested pressure measurements are achieved. Samples may then analyzed using an ASAP 2420 with krypton gas adsorption. It is recommended that these measurements be conducted by Micromeretics Analytical Services, Inc. (One Micromeritics Dr, Suite 200, Norcross, GA 30093). More information on this technique is available on the Micromeretics Analytical Services web sites (www.particletesting.com or www.micromeritics.com), or published in a book, “Analytical Methods in Fine Particle Technology”, by Clyde Orr and Paul Webb.

### Test 9: Dissolution Rate of the Sheet Article

The dissolution rate of dissolvable sheets or solid articles of the present invention is measured as follows:
1. 400 ml of deionized water at room temperature (25° C.) is added to a 1 L beaker, and the beaker is then placed on a magnetic stirrer plate.
2. A magnetic stirrer bar having length 23 mm and thickness of 10 mm is placed in the water and set to rotate at 300 rpm.
3. A Mettler Toledo S230 conductivity meter is calibrated to 1413 μS/cm and the probe placed in the beaker of water.
4. For each experiment, the number of samples is chosen such that a minimum of 0.2 g of sample is dissolved in the water.
5. The data recording function on the conductivity meter is started and the samples are dropped into the beaker. For 5 seconds a flat steel plate with diameter similar to that of the glass beaker is used to submerge the samples below the surface of the water and prevent them from floating to the surface.
6. The conductivity is recorded for at least 10 minutes, until a steady state value is reached.
7. In order to calculate the time required to reach 95% dissolution, a 10 second moving average is firstly calculated from the conductivity data. The time at which this moving average surpassed 95% of the final steady state conductivity value is then estimated and taken as the time required to achieve 95% dissolution.

### Test 10: Measurement of Compressibility

A 25 mm diameter hollow circular hole punch is used to cut a sample disc of about 25 mm in diameter from a flexible, dissolvable, porous sheet of about 8 mm in thickness (as measured by using a Vernier callipers). Said sheet can be a single sheet or a stack of multiple sheets to achieve the desired thickness. The sample disc is stored in an oven with temperature and humidity control capability, for a minimum duration of 4 hours at 25° C. with an equilibrium humidity of 40%.

The compressibility measurements are carried out using a Haake Mars II rheometer, with a PP60 mm plate-plate measuring geometry (model number 222-1271) and an MPC60 measuring plate (model number 222-1550) installed into the rheometer control unit. The base plate temperature of said rheometer is set and controlled at 25° C. throughout the duration of the tests. The rheometer is firstly calibrated before starting the experiments by use of the software to ensure that the zero measurement distance and the zero normal force are both accurately set.

The compression test is then carried out as follows:
1) The 8 mm-thick sample disc is removed from the oven and immediately placed at the center of the MPC60 measuring plate. A thin layer of parafilm is placed on top of the stack to prevent the disc from sticking to the measuring geometry.
2) The measuring geometry is lowered to 4 mm (i.e., the rheometer measurement position) at a speed of 2.5 mm/min as set by the rheometer software. The rheometer measurement position is set at approximately 50% of the original thickness of the sample disc, thereby achieving a Volumetric Compression of about 50% in the sample disc.
3) Once the measuring geometry reaches 4 mm, it is kept stationary for 5 minutes and the pressure force applied thereto by the rheometer is recorded at every second.
4) Once 5 minutes have passed, the measuring geometry is raised, and the sample disc is manually removed.
5) The thickness of the removed sample disc is measured using a Vernier callipers, approximately starting at 5 seconds after the measuring geometry is raised and then every following 30 seconds.

The following parameters are then calculated as follows:

$$50 \% Compression Force ( \frac{N}{{m}^{2}} ) = \frac{\begin{matrix}\begin{matrix}Force measured at 1 second after \\ the rheometer measurement\end{matrix} \\ position is reached\end{matrix}}{\begin{matrix}\begin{matrix}Cross - sectional area of the \\ sample disc in contact with\end{matrix} \\ the measuring geometry plate\end{matrix}}$$

Then, the parameter of Compressibility is determined as 50% Compression Force (N/m²) calculated as above. A low value of 50% compression force as determined above indicates a high compressibility while a high value of 50% compression force as determined above indicates a low compressibility.

### EXAMPLES

### Example 1: Heat-Compressing of Unit Dose Article Containing the Loading Composition

Dissolvable unit dose articles containing the loading compositions (2 sheets and loading composition therebetween) were prepared as follows. Particularly, large flexible, porous, solid sheets (with minimum area 1.0×1.0 m) were prepared according to the method in the Section II: OVERVIEW OF PROCESSES FOR MAKING UNIT DOSE ARTICLES.

Specifically, a wet pre-mixture (i.e., a slurry) containing the ingredients of the solid sheet shown in the following Table 1 and additional water was prepared, to result in a total solids content of around 30% to 45% by weight (i.e., the total water content in the slurry is around 55% to 70% by weight).

TABLE 1

| | Sheet A | | Sheet B | | Sheet C | | Sheet D | | Sheet E | |
|---|---|---|---|---|---|---|---|---|---|---|
| | (Wet) | (Dry) | (Wet) | (Dry) | (Wet) | (Dry) | (Wet) | (Dry) | (Wet) | (Dry) |
| | w/w | w/w | w/w | w/w | w/w | w/w | w/w | w/w | w/w | w/w |
| Materials: | % | % | % | % | % | % | % | % | % | % |
| Polyvinyl alcohol (High | 3.7 | 10 | 4 | 10 | 12.8 | 40 | 10.4 | 28 | 2.9 | 6 |
| Molecular Weight)¹ | | | | | | | | | | |
| Polyvinyl alcohol (Low | 7.4 | 20 | 8.1 | 20 | — | — | — | — | 5.7 | 12 |
| Molecular Weight)² | | | | | | | | | | |
| Glycerin | 4.5 | 12 | 2.4 | 6 | 9.6 | 30 | 10.4 | 28 | 2.9 | 6 |
| Sodium Lauryl Sulfate | — | — | 10.8 | 27 | 1.3 | 4 | — | — | 18.2 | 38 |
| Sodium Laureth 1 Sulfate | 13.4 | 36 | — | — | — | — | — | — | — | — |
| Sodium Laureth 3 Sulfate | 0.8 | 2.2 | 11.3 | 28 | — | — | — | — | — | — |
| Amine Oxide | 3.9 | 10.5 | — | — | — | — | — | — | — | — |
| Alpha-Olefin Sulfonate | — | — | — | — | — | — | — | — | 14.4 | 30 |
| Soap powder | 0.7 | 2 | — | — | — | — | — | — | — | — |
| Diethyloxyester dimethyl | — | — | — | — | — | — | 5 | 13.5 | — | — |
| ammonium chloride | | | | | | | | | | |
| Dodecyl trimethyl | — | — | — | — | — | — | 1.9 | 5 | — | — |
| ammonium chloride | | | | | | | | | | |
| Tetraacetylethylenediamine | — | — | — | — | — | — | — | — | — | — |
| Silicone oil | — | — | — | — | — | — | 5.6 | 15 | — | — |
| Starch | — | — | — | — | — | — | 1.1 | 3 | — | — |
| Perfume Microcapsule | 0.5 | 1.3 | 0.8 | 2 | 6.4 | 20 | — | — | — | — |
| Miscellaneous & Water | 65 | 6 | 62.6 | 7 | 70 | 6 | 66.2 | 7.5 | 56 | 8 |

¹Molecular weight 85,000, Degree of Hydrolysis 87%
²Molecular weight 25,000, Degree of Hydrolysis 87%

The slurry so formed was then aerated and dried in a belt drier by using parameters as shown below to form a solid sheet (i.e., Sheets A to E). Particularly, the belt drier comprises three heating zones in which the three heating zones are configured to simultaneously heat the top and bottom sides of said formed sheet independently at a first top heating temperature (Tₜ₁) and a first bottom heating temperature (T_{b1}) for a first heating duration of from 0.01 minutes to 20 minutes in the Heating Zone 1, heat the top and bottom sides of said formed sheet independently at a first top heating temperature (Tₜ₂) and a first bottom heating temperature (T_{b2}) for a second heating duration of from 0.01 minutes to 20 minutes in the Heating Zone 2, and heat the top and bottom sides of said formed sheet independently at a first top heating temperature (Tₜ₃) and a first bottom heating temperature (T_{b3}) for a third heating duration of from 0.01 minutes to 20 minutes in the Heating Zone 3. The parameters are shown in Table 2 below.

TABLE 2

| Drying method/Temperature | Parameters |
|---|---|
| Belt drier (Step-wise heating) | Slurry Temperature before and during aeration: 60-70° C. |
| Bottom 120-80° C./Top 100-140° C. | Mixing head speed setting for aerator: 300 |
| Sequential Bottom Zones 1 to 3: | Air flow rate setting for aerator: 100 |
| 120° C. 120° C. 80° C. | Slurry Temperature before drying: 60-70° C. |
| Sequential Top Zones 1 to 3: | Drying linear speed: 0.5 to 1.0 m/min |
| 100° C. 120° C. 140° C. | Heating zone area (each): ~3.4 m × ~0.4 m (length × width) |

According to Tests 2, 5 and 6 as described herein, parameters of the solid sheet prepared including Thickness, Density, Basis Weight and Compressibility (i.e. 50% Compression Force) were measured and shown in Table 3 below. Further, Overall Average Pore Diameter (OAPD) of Sheet A and Standard Deviation of OAPD of Sheet A were also measured, i.e. OAPD is 226 μm and SD of OAPD is 104 μm.

TABLE 3

| | Sheet A | Sheet B | Sheet C | Sheet D | Sheet E |
|---|---|---|---|---|---|
| Thickness (mm) | 1.9 | 1.8 | 1.68 | 0.87 | 1.0 |
| Basis weight (g/m²) | 198.9 | 294.4 | 103.5 | 294.4 | 270 |
| Density (g/cm³) | 0.105 | 0.164 | 0.062 | 0.338 | 0.270 |
| Compressibility (N/m²) | 18,178 | 51,376 | 15,722 | 23,189 | 97,138 |

Subsequently, unit dose articles were prepared by loading one of Loading Compositions 1 to 6 as shown in Table 4 within two sheets at a weight ratio of 11 g:5 g (sheets: loading composition) as prepared above by using the conversion method according to the present disclosure. In two approaches, an edge-sealing step and an embossing step are respectively used as the heat-compressing treatment. A first sheet and a second sheet were respectively fed on a belt conveyor sequentially. One of Loading Compositions 1 to 6 was loaded onto the first sheet, and spread evenly between the first and second sheets. Paste was loaded by a loading unit comprising a nozzle and a flattening mechanism while powder was loaded by a loading unit comprising a dispenser and a spreading roller. Finally, the stack of sheets passed through an edge sealing unit comprising an edge sealing roller which comprises a tooling as shown in FIG. 5 (Temperature: 150° C.; Pressure: 2000 psi) so that the stack of sheets was cut into unit dose articles with sealed edge. Alternatively, the stack of sheets passed through an embossing unit which applied heat/pressure at discrete points across the stack (Temperature: 150° C.; Pressure: 2000 psi), and then cut into unit dose articles of which the shape is the same with that shown in FIG. 5. Then, the unit dose articles as prepared were tested to determine if the sealing was successful, i.e., to see if a significant separation between layers or leakage is present. Particularly, the unit dose articles were stored at 25° C. and 40% RH for 48 hours and then checked to see if the edge was still sealed or any significant leakage happened. If the edge was still sealed and no significant leakage happened, the sealing was successful. If the edge was not sealed any more, any separation between layers happened, or any significant leakage happened, the sealing was not successful. The results of sealing are shown in Table 5. The inventors surprisingly found that the sealing is successful both in the approaches of edge-sealing and embossing when the dissolvable porous solid sheet has a relatively high compressibility.

TABLE 4

| | Loading | Loading | Loading | Loading | Loading | Loading |
|---|---|---|---|---|---|---|
| Ingredients | Composition | Composition | Composition | Composition | Composition | Composition |
| (wt %) | 1 (Paste) | 2 (Paste) | 3 (Paste) | 4 (Powder) | 5 (Powder) | 6 (Powder) |
| C12-14 | 20.6 | 22.4 | 30.0 | — | — | — |
| ethoxylated alcohol | | | | | | |
| Linear Alkyl | 20.0 | — | 15.0 | 29.0 | 59.4 | — |
| Benzene Sulfonate | | | | | | |
| Sodium Lauryl | — | — | 25.0 | — | — | — |
| Sulfate | | | | | | |
| Methyl Ester | — | 20.0 | — | — | — | — |
| Sulfonate | | | | | | |
| Silicone oil | — | — | 13.0 | — | — | 33.3 |
| Percarbonate | 55.0 | — | — | 25.0 | — | — |
| Tetraacetylethylenediamine | — | 20.0 | — | 10.0 | — | — |
| Polyalkylene imine | — | 10.0 | — | 3.0 | 10.0 | — |
| polymer A¹ | | | | | | |
| Polyalkylene oxide | 1.0 | — | — | 3.0 | — | — |
| polymer A² | | | | | | |
| Chelant | — | 10.0 | — | 10.0 | — | — |
| Brightener | — | 1.0 | — | 1.0 | — | — |
| Perfume Oil | — | 10.0 | 13.0 | 8.0 | 6.0 | 33.3 |
| Citric Acid | — | — | — | — | 8.0 | — |
| Precipitated Silica | — | — | — | 10.0 | 10.0 | 33.3 |
| Fumed Silica | — | — | 1.0 | — | — | — |
| Miscellaneous & | 3.4 | 6.6 | 3.0 | 1.0 | 6.6 | — |
| Water | | | | | | |

¹Polyethylenimine polymer with 24 EO and 16 PO per —NH with MW 600 from BASF.
²Graft copolymer comprising polyalkylene oxide, N-vinylpyrrolidone and vinyl ester at 50:20:30 ratio with MW 16,800 Dalton from BASF

TABLE 5

| Sealing Success for | | | | | | | | |
|---|---|---|---|---|---|---|---|---|
| Edge-sealing | | Compressibility | Loading Composition | | | | | |
| (Pass/Fail) | | (N/m²) | 1 | 2 | 3 | 4 | 5 | 6 |
| Sheet | A | 18,178 | ✓ | ✓ | ✓ | ✓ | ✓ | ✓ |
| | B | 51,376 | ✓ | ✓ | ✓ | ✓ | ✓ | ✓ |
| | C | 15,722 | ✓ | ✓ | ✓ | ✓ | ✓ | ✓ |
| | D | 23,189 | ✓ | ✓ | ✓ | ✓ | ✓ | ✓ |
| | E | 97,138 | X | X | X | X | X | X |

Sealing Success

| | | |
|---|---|---|
| for Embossing | Compressibility | Loading Composition |

| | | | |
|---|---|---|---|
| (Pass/Fail) | (N/m²) | 4 | 5 |

| | | | | |
|---|---|---|---|---|
| Sheet | A | 18,178 | ✓ | ✓ |
| | B | 51,376 | ✓ | ✓ |
| | C | 15,722 | ✓ | ✓ |
| | D | 23,189 | ✓ | ✓ |
| | E | 97,138 | X | X |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A process for preparing a dissolvable unit dose sheet article, comprising the steps of:
a) providing a first flexible, dissolvable, porous sheet, a second flexible, dissolvable, porous sheet and a loading composition in a form of paste or powders in which each of the first and second sheets comprises a water-soluble polymer and a surfactant;
wherein each of the first and the second sheets is characterized by:
a Percent Open Cell Content of from 80% to 100%,
an Overall Average Pore Size of from 100 μm to 2000 μm, and
a Compressibility of less than 90,000N/m²;
b) applying the loading composition on a surface of the first sheet;
c) arranging the first and second sheets into a stack so that the loading composition is contained between the first and second sheets; and
d) heat-compressing said stack of sheets to form the dissolvable unit dose article.

2. The process of claim 1, wherein each of the first and the second sheets is characterized by a Compressibility of from 1,000 N/m² to 90,000 N/m².

3. The process of claim 1 or 2, wherein Step d) is performed under a temperature of from 50° C. to 200° C., a pressure of from 100 psi to 20000 psi, and a contacting time of from 0.02 s to 10 s, and/or wherein Step d) is performed on at least 5% of the surface area of the sheet.

4. The process of claim 1, wherein the loading composition is in a form of non-aqueous paste and comprises a non-aqueous liquid carrier, solid particles and a polyalkylene polymer,
wherein said non-aqueous paste comprises:
from 1% to 99% of a non-aqueous liquid carrier by total weight of said non-aqueous paste; and/or
from 1% to 99% solid particles by total weight of said non-aqueous paste; and/or
from 0.5% to 50% of a polyalkylene polymer by total weight of said non-aqueous paste.

5. The process of claim 4, wherein said non-aqueous liquid carrier is selected from the group consisting of polyethylene glycol, polypropylene glycol, silicone, fatty acid, perfume oil, a non-ionic surfactant, an organic solvent and any combinations thereof, wherein said non-aqueous liquid carrier comprises a non-ionic surfactant that is selected from the group consisting of C₆-C₂₀ linear or branched alkylalkoxylated alcohols (AA) having a weight average degree of alkoxylation ranging from 5 to 15; and/or
wherein said solid particles comprise an oxidative dye compound, a pH modifier and/or a buffering agent, a radical scavenger, a chelant, a warming active, a color indicator, an anionic surfactant, an enzyme, a bleaching agent, an effervescent system and any combinations thereof, wherein said solid particles comprises C₆-C₂₀ linear alkylbenzene sulphonate (LAS) surfactant, percarbonate salts, perborate salts, persulfate salts, tetraacetylethylenediamine (TAED), oxybenzene sulphonates, caprolactams, or any combinations thereof; and/or
wherein said polyalkylene polymer is selected from a group consisting of polyalkylene imine polymer, polyalkylene oxide polymer and any combinations thereof, wherein said polyalkylene polymer is a polyalkylene graft copolymer comprising a) polyalkylene oxide component as a graft base, and b) polyvinyl ester component as side chains, and/or c) polyvinylpyrrolidone as side chains.

6. The process of claim 1, wherein the loading composition is in a form of powders which are characterized by a bulk density of from 250 g/l to 500 g/l,
wherein the powders are characterized by a mean particle size of from about 200 to about 600 microns.

7. The process of claim 6, wherein said powders comprises an anionic surfactant which is selected from the group consisting of C₆-C₂₀ linear alkylbenzene sulfonate (LAS), a C₆-C₂₀ linear or branched alkylalkoxy sulfates (AAS) having a weight average degree of alkoxylation ranging from 0.5 to 10, a C₆-C₂₀ linear or branched alkyl sulfates (AS) and any combinations thereof.

8. The process of claim 1, wherein an aqueous liquid is sprayed on a surface of the second sheet before the Step c) in which the surface of the second sheet is located to be adjacent to the first sheet.

9. The process of claim 1, wherein the heat-compressing is selected from the group consisting of edge-sealing, embossing and any combinations thereof,
wherein the heat-compressing is edge-sealing under a temperature of from 90° C. to 170° C., a pressure of from 5000 psi to 15000 psi, and a contacting time of from 0.02 s to 10 s.

10. The process of claim 1, wherein the process further comprises: providing one or more additional flexible, dissolvable, porous sheets onto the stack of the first and the second sheets before heat-compressing, wherein each of the additional sheets is characterized by:
a Percent Open Cell Content of from 80% to 100%,
an Overall Average Pore Size of from 100 μm to 2000 μm, and
a Compressibility of less than 90,000N/m².

11. The process of claim 1, wherein the process is a continuous process which is performed on a conveying belt.

12. The process of claim 11, wherein Step b) is conducted by a loading unit comprising a nozzle and a flattening mechanism or a dispenser and a spreading roller; and/or
wherein Step c) is conducted by one or more rollers; and/or
wherein Step d) is conducted by an edge-sealing roller and optionally a transferring roller; and/or
wherein Step d) is conducted by an embossing roller and a cutting roller as well as optionally and a transferring roller.

13. The process of claim 1, wherein the weight ratio of the sheets and the loading composition in the dissolvable unit dose article is between 1000 and 0.1.

14. The process of claim 1, wherein each of the sheets is characterized by:
a Percent Open Cell Content of from 85% to 100%; and/or
an Overall Average Pore Size of from 150 μm to 1000 μm; and/or
an Average Cell Wall Thickness of from 5 μm to 200 μm; and/or
a final moisture content of from 0.5% to 25% by weight of said solid sheet article; and/or
a thickness of from 0.6 mm to 3.5 mm; and/or
a basis weight of from 50 grams/m² to 500 grams/m²; and/or
a density of from 0.05 grams/cm³ to 0.5 grams/cm³; and/or
a Specific Surface Area of from 0.03 m²/g to 0.25 m²/g.

15. A product in the form of a dissolvable unit dose sheet article, wherein the product is selected from the group consisting of laundry detergent products, fabric softening products, hand cleansing products, hair shampoo or other hair treatment products, body cleansing products, shaving preparation products, dish cleaning products, personal care substrates containing pharmaceutical or other skin care actives, moisturizing products, sunscreen products, beauty or skin care products, deodorizing products, oral care products, feminine cleansing products, baby care products, fragrance-containing products and any combinations thereof,
wherein the product comprises a plurality of water-soluble sheets arranged in a stack, wherein each of the water-soluble sheets comprises a water-soluble polymer and a surfactant,
wherein each of the water-soluble sheets is characterized by: a Percent Open Cell Content of from 80% to 100%; an Overall Average Pore Size of from 100 μm to 2000 μm; and a Compressibility of less than 90,000N/m²;
wherein the product has a sealed edge and/or an embossing.

16. A system for preparing dissolvable unit dose sheet articles comprising two or more flexible, porous, dissolvable solid sheets and a loading composition in a form of paste or powders contained within said two or more sheets,
wherein each of the sheets comprises a water-soluble polymer and a surfactant;
wherein each of the sheets is characterized by:
a Percent Open Cell Content of from 80% to 100%,
an Overall Average Pore Size of from 100 μm to 2000 μm, and
a Compressibility of less than 90,000N/m²,
wherein the system comprises:
a belt conveyor on which two or more flexible, porous, dissolvable solid sheets are sequentially fed;
a loading unit which is configured to load a loading composition within said two or more sheets, comprising a nozzle and a flattening mechanism or a dispenser and a spreading roller; and
an edge-sealing unit or an embossing unit.
